(19) United States
(12) Patent Application Publication
(10) Pub. No.: US 2025/0262313 A1
(43) Pub. Date: Aug. 21, 2025

(54) Targeted Ligand-Payload Based Drug Delivery for Cell Therapy

(71) Applicant: Purdue Research Foundation, West Lafayette, IN (US)

(72) Inventors: Philip S. Low, West Lafayette, IN (US); Madduri SRINIVASARAO, West Lafayette, IN (US); Boning Zhang, West Lafayette, IN (US)

(21) Appl. No.: 19/194,112

(22) Filed: Apr. 30, 2025

Related U.S. Application Data
(62) Division of application No. 16/486,632, filed on Aug. 16, 2019, now Pat. No. 12,343,403.
(60) Provisional application No. 62/460,118, filed on Feb. 17, 2017.

(51) Int. Cl. A61K 47/66; A61K 38/00; A61K 40/10; A61K 40/11; A61K 40/31; A61K 40/42; A61K 47/69; C07K 14/725; C12N 9/90; C12N 15/62

(52) U.S. Cl. CPC A61K 47/66; A61K 40/10; A61K 40/11; A61K 40/31; A61K 40/4211; A61K 47/6901; C07K 14/7051; C12N 9/90; C12N 15/62; A61K 38/00; A61K 2239/31; A61K 2239/38; A61K 2239/48; C07K 2319/03; C07K 2319/20; C07K 2319/912; C12Y 502/01008

(57) ABSTRACT

A drug delivery platform providing flexible fine tune of cell therapy is disclosed herein. Particularly, an engineered fusion protein is coupled with a high affinity ligand carrying at least one payload of drug to be internalized by the transplanted cell to observe or regulate transplanted cell therapy effects.

### CROSS REFERENCE

This application is a divisional of and claims the benefit of priority of U.S. application Ser. No. 16/486,632, filed Aug. 16, 2019, which is a U.S. National Stage Filing under 35 U.S.C. § 371 from International Application No. PCT/US2018/018557, filed on Feb. 17, 2018, and published as WO2018/152451 on Aug. 23, 2018, which claims the benefit of U.S. provisional application 62/460,118 under 35 U.S.C. § 119(e), filed on Feb. 17, 2017; the benefit of priority of each of which is hereby claimed herein, and which applications and publication are hereby incorporated herein by reference in their entireties.

### INCORPORATION BY REFERENCE OF SEQUENCE LISTING

This application contains a Sequence Listing which has been submitted electronically in ST26 format and hereby incorporated by reference in its entirety. Said ST26 file, created on Apr. 30, 2025, is named 1165083US2.xml and is 101,958 bytes in size.

### FIELD OF INVENTION

This disclosure provides a drug delivery platform for cell therapy. Particularly, an engineered protein is coupled with a high affinity ligand carrying at least one payload of drug to be internalized by the transplanted cell through the engineered protein to regulate transplanted cell therapy effects.

### BACKGROUND

In the last few decades, great advances have been made in this field regarding the cell types, delivery methods and suitable diseases models. In terms of cell types, current cell therapies can be roughly categorized as chimeric antigen receptors (CARs), cell for tumor model and stem cell based regenerative medicine.

CAR T also known as chimeric T cell receptors, chimeric immunoreceptors or artificial T cell receptors, enable immune effector cells (usually T cells or NK cells) to recognize target cells with corresponding antigen and exercise their cytotoxic activity. The emergence and development of CAR-T technology provides promises to certain types of cancers, which turns CAR-T into a superstar in the field of both biomedical research and clinical studies.

Regenerative medicine is a game-changing area of medicine with the potential to fully heal damaged tissues and organs, offering solutions and hope for people who have conditions that today are beyond repair. Advances in developmental and cell biology, immunology, and other fields have unlocked new opportunities to refine existing regenerative therapies and develop novel ones.

Stem cells have the ability to develop—through a process called differentiation—into many different types of cells, such as skin cells, brain cells, lung cells and so on. Stem cells are a key component of regenerative medicine, as they open the door to new clinical applications.

A variety of stem cells, including adult and embryonic stem cells may be used in regenerative medicine. In addition, various types of progenitor cells, such as those found in umbilical cord blood, and bioengineered cells called induced pluripotent stem cells are used in regenerative medicine. Each type has unique qualities, with some being more versatile than others.

Many of the regenerative therapies under development begin with the particular patient's own cells. For example, a patient's own skin cells may be collected, reprogrammed in a laboratory to give them certain characteristics, and delivered back to the patient to treat his or her disease.

Although the anti CD19 CAR T has received great success in clinical applications for leukemia treatment, lethal side effects such as the cytokine storm generated from the fast lysis of tumor cells, as well as the killing of normal CD19+ B cells by the fast proliferating anti-CD19 CAR T cell requires finer control of the CAR T cell. In stem cell based regenerative therapy, efforts have been put to better understand the differentiation process and trophic roles of the transplanted cells in the target tissue. Meanwhile, these processes can be potentially altered by some small molecule drugs that specifically delivered to the stem cell to further contribute to the regeneration of the target tissue.

Another long lasting concern about the CAR T cells as well as other stem cell based regenerative therapy is the tumorigenic potential of these transplanted cells. In summary, it will be ideal to have a private doorway to control the activity of the transplanted cell, either CAR T cell or stem cell, after they are being transplanted.

### SUMMARY OF THE INVENTION

This disclosure provides a drug delivery platform for fine tuning cell therapy. The drug delivery system comprises:
a. an engineered protein on a target cell for transplant, wherein the fusion protein comprises a first component and a second component, the first component and the second component are connected by a peptide linker, the first component is a non-membrane protein, the second component is a membrane anchored peptide or protein;
b. at least one small ligand conjugated to a linker, wherein the at least one small ligand has intrinsic high affinity to at least one component of the engineered protein; and
c. at least one payload of drug conjugated to the linker, wherein the payload of drug is associated with the target cell when the small ligand binds to at least one component of the engineered protein.

In some embodiment, the aforementioned drug delivery platform has a payload of drug of imaging agent. Such imaging agent may be sleeted from from the group consisting of fluorescent dye rhodamine, fluorescein, and S0456. Alternatively, such imaging agent is selected from the group consisting of radioisotope chelating imaging moieties, EC 20 chelating head, NOTA and DOTA.

In some embodiment, the aforementioned drug delivery platform has a payload of drug of cytotoxic drug. Such cytoxic drug may be selected from the group consisting of tubulysin, DM1, DM4, and an auristatin.

In some embodiment, the aforementioned drug delivery platform has a payload of drug of a modulator of gene expression.

In some embodiment, the aforementioned drug delivery platform has a payload of drug of modulator of the cell's activity.

In some embodiments, the aforementioned modulator may be selected from the group of Dasatinib, MEK1/2 inhibitor, and PI3K inhibitor; group of HDAC inhibitor, kinase inhibitor and metabolic inhibitor; group of GSK3 beta inhibitor, MAO-B inhibitor and Cdk5 inhibitor.

In some embodiments, the aforementioned modulator is a phosphatase inhibitor, an RORγt agonist or an siRNA mi181a1.

In some embodiments the aforementioned payload of drug is a phosphatase inhibitor, including but not limited to inhibitors against SHP1/2, TC-PTP.

In some embodiment, the aforementioned payload of drug in the drug delivery platform is further internalized by the target cell when the small ligand binds to at least one component of the engineered protein.

In some embodiment, the aforementioned drug delivery platform has a releasable linker to connect the small ligand and the payload drug. The linker can be selected from the group consisting of

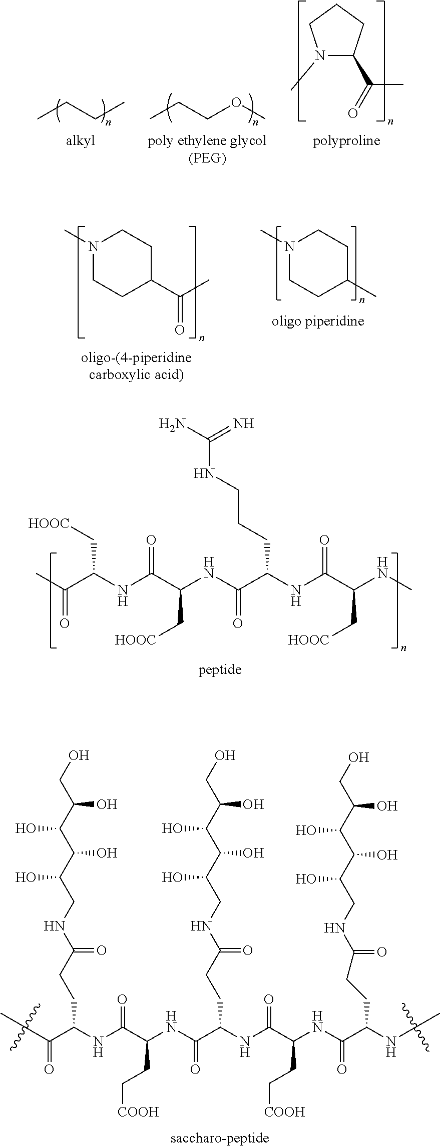

In some embodiment, the aforementioned engineered protein components are are selected from the group consisting of Folate Receptor alpha (FRa), Folate Receptor beta (FRb), Urokinase receptor (uPAR), FK506 binding protein (FKBP), dihydrofolate reductase (DHFR), Single Chain Fragment Variable against Fluorescein isothiocyanate (scFv against FITC), and Single Chain Fragment Variable against dinitrophenol (scFv against DNP).

In some embodiment, the aforementioned small ligand is selected from the group consisting of

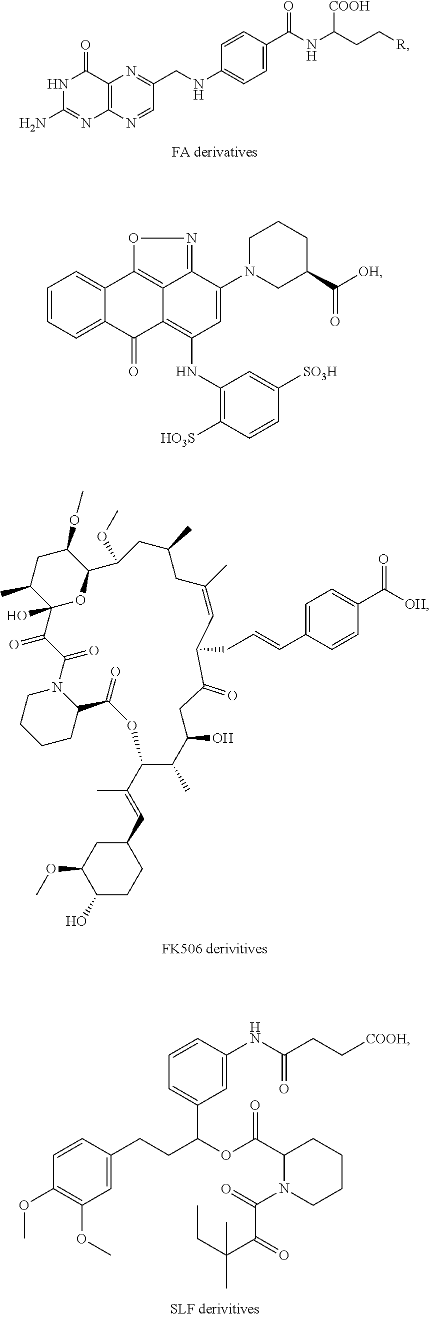

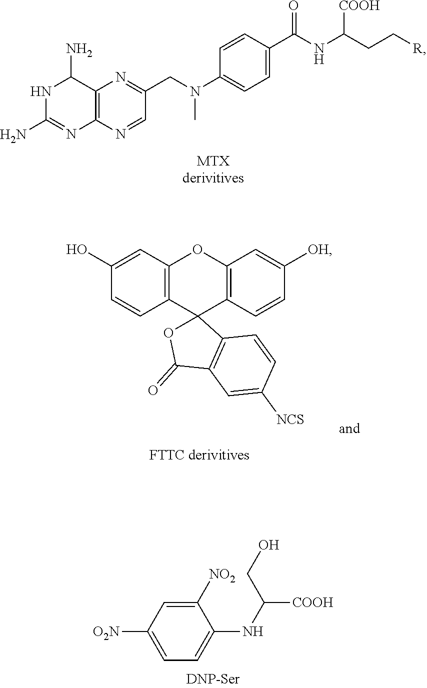

In some embodiment, the aforementioned drug delivery platform has the first component as FKBP, the second component is a peptide that confers a glycosylphosphatidyl inositol (GPI) anchor on the first component, and the small ligand is FK506 or its derivative. In some embodiment, the FK506 derivative abolishes Calcineurin binding site.

In some embodiment, the aforementioned second component is a full length or truncated Folate Receptor (FR).

In some embodiment, the aforementioned drug delivery platform has at least one segment of flexible peptide linker SGGGS to connect the first component and the second component of the engineered protein.

In some embodiment the aforementioned drug delivery platform comprises an engineered protein selected from the group consisting of SEQ ID NOS: 1-2 (amino acid sequence for mouse FKBP-FRa and amino acid sequence for human FKBP-FRa respectively).

In some embodiment the aforementioned drug delivery platform comprises an engineered protein selected from the group consisting of SEQ ID NOS: 12-15.

In some embodiment the aforementioned target cell for transplant is an immune cell. For example, the immune cell can be a NK cell or a Chimeric Antigen Receptor T (CAR T) cell. Such CAR T cell may be expressing amino acid sequence selected from SEQ ID NOS: 3-4.

In some embodiment the aforementioned drug delivery platform has the small ligand conjugate with formula I.

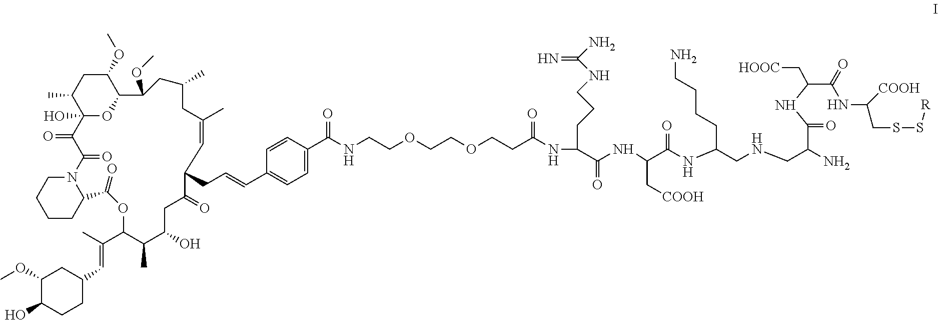

In some embodiment the aforementioned drug delivery platform has a target cell for transplant as CAR T cell expressing SEQ ID NO:3 (amino acid sequence for mouse antiCD19 CAR T construct) or SEQ ID NO:4 (amino acid sequence for human antiCD19 CAR T construct).

In some embodiment the aforementioned small ligand is further conjugated to a fluorescent dye or radioactive probe for tracking the drug internalization.

In some embodiment the aforementioned drug delivery platform comprises a small ligand that is further conjugated to a regulator of endogenous gene expression of a regulator of a transduced transgene expression.

In some embodiment the aforementioned drug delivery platform, the target cell for transplant is a stem cell, a progenitor cell, or a transplanted cell designed to synthesize a biochemical that is deficient in a patient. This disclosure further provides a CAR T cell comprising a construct expressing amino acid sequences selected from SEQ ID NOS: 12-15.

This disclosure further provides a DNA construct encoding an amino acid sequence selected from the group consisting of SEQ ID NOS: 12-15.

This disclosure further provides a DNA construct encoding a FKBP-FRa fusion receptor comprising any of SEQ ID NOS: 1-2 operably linked to an EF1a promoter in a expression vector. In some embodiment, such expression vector is pWPI having SEQ ID NO:5.

This disclosure further provides a DNA construct comprising any of SEQ ID NOS: 6-8.

This disclosure further provides a transplanted cell comprising insert genes hFKBP-FR (SEQ ID NO:7) and human antiCD19 CAR (SEQ ID NO:9).

This disclosure further provides a transplant cell comprising insert genes mFKBP-FR (SEQ ID NO:8) and mouse antiCD19 CAR (SEQ ID NO:10).

This disclosure further provides a method to modulate cell therapy effect. The method comprises:
a. Identifying a target cell for transplant, wherein the transplanted target cell has a cell therapy function;
b. Providing an engineered fusion protein on the surface of the target cell for transplant, the fusion protein comprises a first component and a second component, the first component and the second component are connected by a flexible peptide linker, the first component is a non-membrane protein, the second component is a Glyclosylphosphatidyl inositol (GPI) anchored peptide or protein;
c. Providing a payload of drug conjugate to the target cell, wherein the payload of drug is conjugated to a small ligand through a linker, and optionally conjugated to a fluorescent dye, wherein the small ligand binds to at least one component of the engineered fusion protein with high affinity and is internalized by the target cell together with the payload of drug;
d. releasing the drug within the target cell to modulate the target cell therapy function.

In some embodiment, the aforementioned cell therapy function is to provide optically guided surgery to a subject.

In some embodiment, the aforementioned cell therapy function is to control the target cell proliferation.

In some embodiment, the aforementioned cell therapy function is to execute cytotoxicity to the target cell engaged cancer cell.

In some embodiment, the aforementioned transplanted target cell is an immune cell. For example, the target cell is a CAR T cell.

In some embodiment, the aforementioned transplanted target cell is a stem cell, a progenitor cell or a transplanted cell designed to synthesize a biochemical that is deficient in a patient.

In some embodiment, the aforementioned payload of drug is an imaging agent selected from fluorescent dye of Rhodamine and FITC, or a radioisotope imaging agent selected from EC20 chelating head, NOTA and DOTA.

In some embodiment, the aforementioned payload of drug is a cytotoxic drug selected from the group consisting of Tubulysin, DM1, DM4 and auristatin.

In some embodiment, the aforementioned payload of drug is a modulator of gene expression selected from kinase inhibitors consisting of Dasatinib, MEK1/2 inhibitor and PI3 Kinase inhibitor, or an siRNA of mi181a1.

In some embodiment, the aforementioned transplanted target cell comprises a fusion protein selected from the group consisting of SEQ ID NOS: 12-15.

In some embodiment, the aforementioned engineered protein components are selected from the group consisting of FRa, FRb, uPAR, FKBP, DHFR, scFv against FITC, and scFv against DNP.

In some some embodiment, the aforementioned small ligand is selected from the group consisting of

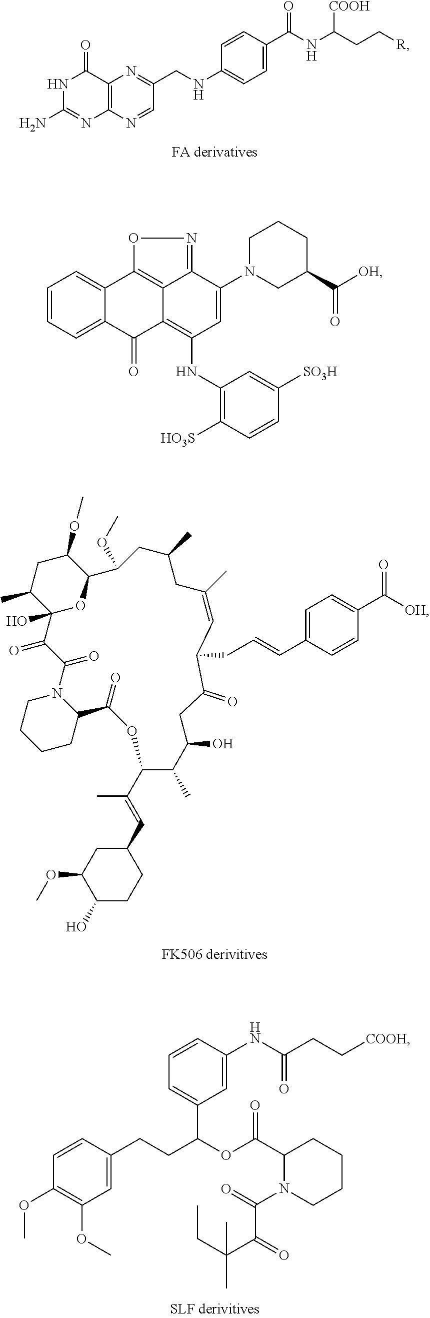

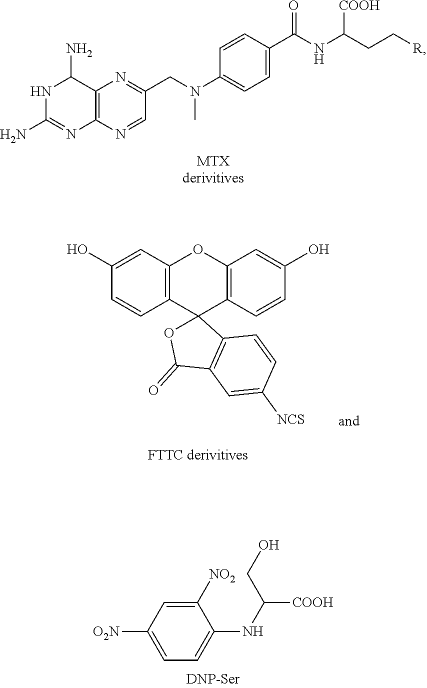

In some embodiment, the aforementioned linker to connect the small ligand and the payload drug is selected from the group consisting of

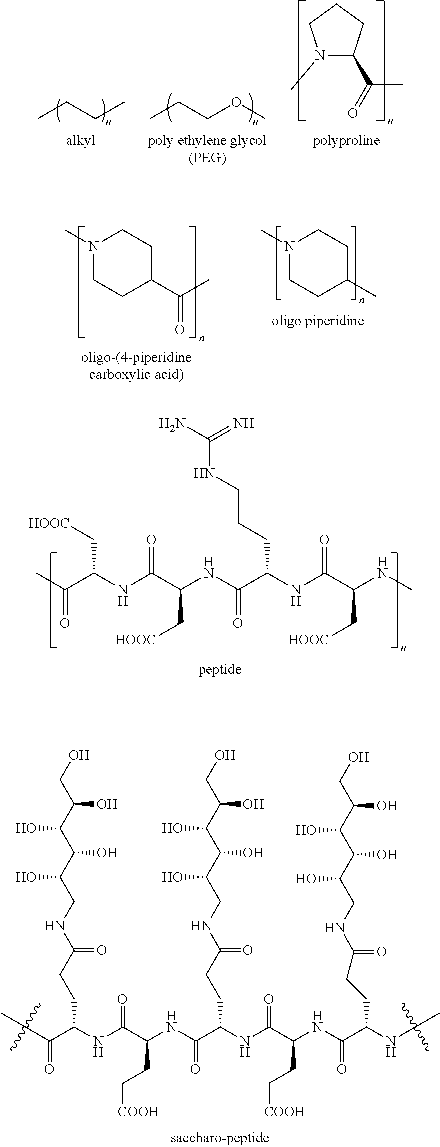

In some embodiment, the aforementioned transplanted target cell comprises an engineered FKBP-LINKER-FRa fusion protein selected from group consisting of SEQ ID NO:1 and SEQ ID NO:2.

In some embodiment, the aforementioned transplanted target cell is CAR T cell comprising an engineered antiCD19 CAR T construct selected from group consisting of SEQ ID NO:3 and SEQ ID NO:4.

In some embodiment, the aforementioned drug conjugate is FK506-releasable linker comprising formula I, wherein the binding domain of FK506 has an affinity to FKBP of about 4 pM to about 100 pM.

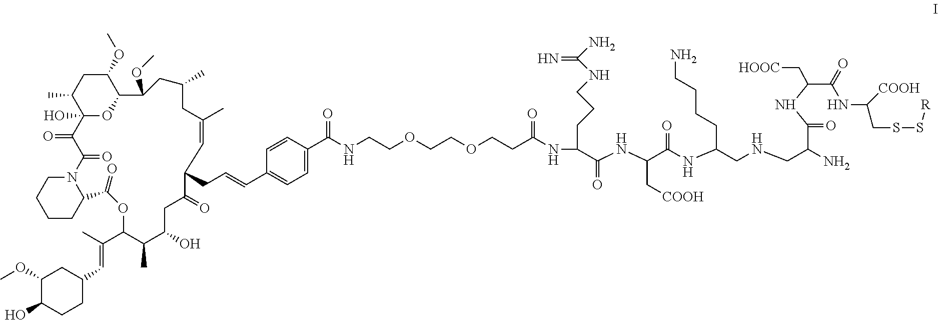

In some embodiment the aforementioned transplanted target cell is a CAR T cell and the drug conjugate is selected from the group consisting of GSK3b inhibitor, MAPK inhibitor to control excessive cytokine storm of transplanted CAR T cell.

In some embodiment the aforementioned transplanted target cell is a CAR T cell and the drug conjugate is a modulator designed to control unwanted T cell proliferation.

In some embodiment the aforementioned transplanted target cell is a stem cell or progenitor cell and the drug conjugate is GSK3b inhibitor to boost bone fracture repair

In some embodiment the aforementioned transplanted target cell is a stem cell, a progenitor cell or a transplanted cell designed to synthesize a biochemical that is deficient in a patient; and the drug conjugate is selected from the group consisting of MAO-B inhibitor and cdk5 inhibitor to treat Parkinson disease or other neurodegenerative disease.

In some embodiment the aforementioned transplanted target cell is a NK cell and the drug conjugate is a RORγt agonist to control Th17 cell mediated immune responses.

These and other features, aspects and advantages of the present invention will become better understood with reference to the following figures, associated descriptions and claims.

### BRIEF DESCRIPTION OF THE DRAWINGS

FIG. 1A. overview of the FKBP-FRa and FK506-payload based drug delivery platform for cell therapy.

FIG. 1B. Graphic illustration of the secret pathway platform for CAR T cells delivery of payload.

FIG. 2A. left: chemical structure of FK506 with FKBP binding sites (yellow) and derivatized site (red) highlighted. Right: co-crystal structure of ternary complex of a calcineurin A fragment (green), calcineurin B (cyan), FKBP12 (purple) and the FK506 (yellow), PDB: 1TCO.

FIG. 2B. Various combination options of two modules in an engineered fusion protein and their respective ligand choices, with potential derivatization sites highlighted.

FIG. 3: left: negative and positive regulation of the CAR T cell activity, adapted from The quest or spatio-temporal control of CAR T cells, Sun J. etc. 2015. Right: mechanism of AP1903 (FK506 dimer) induced FKBP-caspase9 mediated apoptosis and the structure of AP1903, adapted from Inducible Apoptosis as a Safety Switch for Adoptive Cell Therapy, Malcolm K. B. etc. 2011.

FIG. 4A. pWPI expression vector map with FKBP-FRa insert (hFRa 1-24: red, FKBP: yellow, hFRa 25-258: red).

FIG. 4B. FKBP-FRa transduced K562 cells show a higher band around 50 kDa (37 kDa for FRa plus 12 kDa for FKBP) compare to FRa positive KB cells and non-transduced K562 cells.

FIG. 4C. payload carrier construct and CAR T construct design.

FIG. 4D. The construct design of FKBPFR3GS (noted as FF3). From N terminal to C terminal, it has 1-24 aa of human FRa as the signal peptide, human FKBP protein, three Gly-Ser linker and then 25-258 aa of human FRa. In FKBPFR1GS (noted as FF1), the three Gly-Ser liner of FF3 is substituted with one Gly-Ser linker with other parts unchanged.

FIG. 4E. The construct design of 4m5.3FR. From N terminal to C terminal, it has hCD8 signal peptide, scFv of 4M5.3 antibody against FITC, GS linker, 25-258aa of human Fra.

FIG. 5. Interference between the FR and FKBP in FKBPFR1GS fusion receptor. Binding of Folate acid in FKBPFR1GS fusion protein blocks the binding of FK506-Rhodamine at as low as 0.01 nM and totally abolish FK506-Rhodamine binding at 50 nM.

FIG. 6. FKBPFR1GS jurkat cell shows decreased FK506-Rhodamine intensity after binding to OTL38. FRET from FK506-Rhodamine (donor) to OTL38 (FA-S0456, acceptor, ex/em:774/794 nm) within the fusion receptor indicates the interaction between FR and FKBP.

FIG. 7. Increasing the linker length between FKBP and FR significantly lowers the interference between the two parts. Compare to FF1 (1GS between FKBP and FR), FF3 (3GS between FKBP and FR) preserves the binding of FK506-Rhodamine in the presence of 10 nM FA, which is comparable to the physiological concentration of FA in human body.

FIG. 8. PI-PLC treatment releases the GPI anchored fusion receptor FF3. Jurkat T cell with FF3 fusion receptor shows saturated binding with 20 nM FA-FITC (EC17), while after 5 mU PI-PLC or 50 mU PI-PLC treatment, the FA-FITC loses binding to the cell, which indicates the release of the GPI anchored FF3 fusion receptor.

FIG. 9. FA-Rhodamine binding curve in FKBPFR3GS fusion receptor. FKBPFR3GS fusion receptor that stably expressed on human T cell can bind to folate acid derivative (FA-Rhodamine) with high affinity (Kd=0.95 nM), which is comparable to the affinity of FA-Rhodamine in FR+KB cell (Kd around 1 nM). Therefore, the binding property of FR in the fusion receptor is preserved.

FIG. 10. FK506-Rhodamine binding curve in FKBPFR3GS fusion receptor. FKBPFR3GS fusion receptor that stably expressed on human T cell is able to bind to FK506 derivative (FK506-Rhodamine) with high affinity (Kd=3.93 nM), which means the binding property of FKBP in the fusion receptor is preserved.

FIG. 11. SLF-FITC binds to FKBPFR3GS fusion receptor with relatively high binding affinity (Kd=62 nM), while competition with free SLF (100×, preincubation) blocks this binding. SLF, a mimic of FK506, presents a 10 times lower binding affinity to FKBPFR fusion receptor, compare to the parent ligand FK506, which is consistent with previous report.

FIG. 12. FA-Rhodamine binding curve in 4M5.3FR fusion receptor. FA-Rhodamine can binds to 4M5.3FR fusion receptor that stably expressed on human T cell with high affinity (Kd=2.25 nM), which is comparable to the affinity of FA-Rhodamine in FR+KB cell (Kd around 1 nM). Therefore, FR binding property is preserved in 4M5.3FR fusion receptor.

FIG. 13. FITC-AF647 binding curve in 4M5.3FR fusion receptor. FITC-AF647 can binds to 4M5.3FR fusion receptor that stably expression on human T cell with high affinity (Kd=8.03 nM). 100× comp indicates free FITC sodium. The binding property of scFv 4M5.3 with FITC is preserved in 4M5.3FR fusion receptor.

FIG. 14. FA-Tubulysin is able to mediate a receptor specific killing effect against FF3+ human T cell. Compensation with FA (100× preincubation with FA) blocks the effect. This implies the successful internalization and release of the free drug Tubulysin through the FF3 fusion receptor system.

FIG. 15. FA-Tubulysin specifically kill the hFF3+ population in a mixed human T cell culture. Absolute number of hFF3+ cell decrease as the FA-Tub concentration increases, while hFF3-cells are killed also through released drugs and bystander effect at high concentration.

FIG. 16. SLF-Tub specifically kill the hFF3+ Jurkat cells with a IC₅₀—138 nM. This indicates the successful internalization of SLF-Tub by the FKBPFR3GS fusion receptor and the release of the Tubulysin inside the cell.

FIG. 17. Both FITC-DM4 and FITC-Tub can specifically kill the 4M5.3FR+ human T cells, while FITC-Tub has a higher IC₅₀. Compensation of free FITC sodium (100× preincubation) blocks the receptor mediated killing effect. This implies the successful internalization and release of FITC-cytotoxic drug into T cell through 4M5.3FR fusion receptor.

FIG. 18. FITC-Tubulysin specifically kill the 4M5.3FR+ population in a mixed human T cell culture. Absolute number of 4M5.3FR+ cell decrease as the FITC-Tub concentration increases, while the 4M5.3FR− cells are killed also through released drugs and bystander effect at high concentration.

FIG. 19, FITC-DM4 specifically kill the 4M5.3FR+ population in a mixed human T cell culture. Absolute number of 4M5.3FR+ cell decrease as the FITC-DM4 concentration increases, while the 4M5.3FR− cells are killed also through released drugs and bystander effect at high concentration.

FIG. 20. Dasatinib (Lck inhibitor) and Ibrutinib (ITK inhibitor) at 10 nM concentration decrease the lysis effect of antiCD19 CAR T cell (FMC63 CAR T, Effector) against CD19+ Raji tumor cell (Target). Two Effector: Target ratio (E:T) have been tested. Normal T cell and antiCD19 CAR T with CD19−K562 cell were used as negative control.

FIG. 21. FITC-Dasatinib can decrease the lysis effect of FMC63+4M5.3FR+hT cell towards Raji cell. This implies the successful internalization and release of FTTC-Dasatinib into T cell through 4M5.3FR fusion receptor and the release of Dasatinib into T cell.

FIG. 22. TC-PTP inhibitor at 100 nM concentration decrease the co-inhibitor molecule population in exhausted antiCD19 CAR T cell (generated by 7 times of stimulation with CD19+Raji cells, see detailed procedure below). Both PD-1, LAG3 and double positive population decreases upon treatment. This implies that phosphatase inhibitors, like TC-PTP inhibitor may be used as a payload for the secret gateway platform for rejuvenating the exhausted CAR T cell.

TABLE 1
Potential applications of the FKBP-FRa cell
therapy platform and the corresponding payload.
SEQUENCE LISTINGS

SEQ ID NO: 1 Amino acid sequence for mouse FKBP-FRa
SEQ ID NO: 2 Amino acid sequence for human FKBP-FRa
SEQ ID NO: 3 Amino acid sequence for mouse antiCD19 CAR T
construct
SEQ ID NO: 4 Amino acid sequence for human antiCD19 CAR T
construct
SEQ ID NO: 5 vector pWPI for human T cell transduction
SEQ ID NO: 6 pMP71 gb NoIlEcoRI mouse antiCD19 for mouse T
cell transduction
SEQ ID NO: 7 pWPI-FRa 1-24 FKBP FRa
SEQ ID NO: 8 pWPI niFKBP-mFRa SGGGS
SEQ ID NO: 9 pHR EcorI hAnti cdl9 1D3 myc hinge cd28 cd3zeta
SEQ ID NO: 10 pWPI pmei mAnti cd19 1D3 myc hinge cd28 cd3zeta
SEQ ID NO: 11 FKBP-1SG-FR with GPI anchor amino acid sequence
SEQ ID NO: 12 FKBP-3SG-FR with GPI anchor amino acid sequence
SEQ ID NO: 13 4M5.3-FR with GPI anchor amino acid sequence
SEQ ID NO: 14 FMC63-T2A-FKBP3SGFR
SEQ ID NO: 15 FMC63-T2A-4M5.3SGFR
SEQ ID NO: 16 FRb with signal peptide
SEQ ID NO: 17 uPAR with signal peptide
SEQ ID NO: 18 DHFR
SEQ ID NO: 19 scFv against FITC: 4M5.3(Kd = 200 pM)
SEQ ID NO: 20 scFv against FITC 4D5Flu (Kd = 10 nM)
SEQ ID NO: 21 scFv against DNP SPE7

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

This disclosure provides a novel platform for controlling the transplanted cell activity by genetically incorporating a fusion receptor on the transplanted cell surface. These transplanted cells will then be specifically targeted by a small molecule ligand conjugated-drug payload, using the intrinsic high affinity between the small molecule ligand and the fusion receptor on the transplanted cell surface. One part of the fusion receptor is responsible for internalizing the conjugate and the payload will be released through a releasable linker once it is inside of the transplanted cells. Depending on the transplanted cell type and the desired regulation to be imposed on the transplanted cells, the drug payload can be various functions. By changing the payload in the conjugate, for example, as cytotoxic drug or kinase inhibitors, the drug payload may be used to control the multiple aspects of the transplanted cells, such as proliferation, differentiation or cytokine release profile.

The peptidyl-proline isomerase (PPIases) family consists of FK506-binding protein (FKBP), cyclophilins and parvulins. In human, there are 18 FKBPs, 24 cyclophilins and 3 parvulins. Among these, FKBP51 and FKBP52 share high to moderate binding affinity of FK506, KD^{FK506}≈104 nM and KD^{FK506}≈23 nM, respectively, comparing to FKBP12 (KD^{FK506}≈0.2 nM). Additionally, none of these two FKBPs are expressed on the cell membrane, resulting in little cross binding activity in our system. Efforts have also been made to design synthetic ligands that have higher affinity to FKBP12^{F36V} than FKBP^{WT}, as well as to FKBP51^{F67V}, which preserve the overall structure of the wild type proteins. All the homolog and mutated proteins together with their ligands mentioned above, can be adapted to this disclosure.

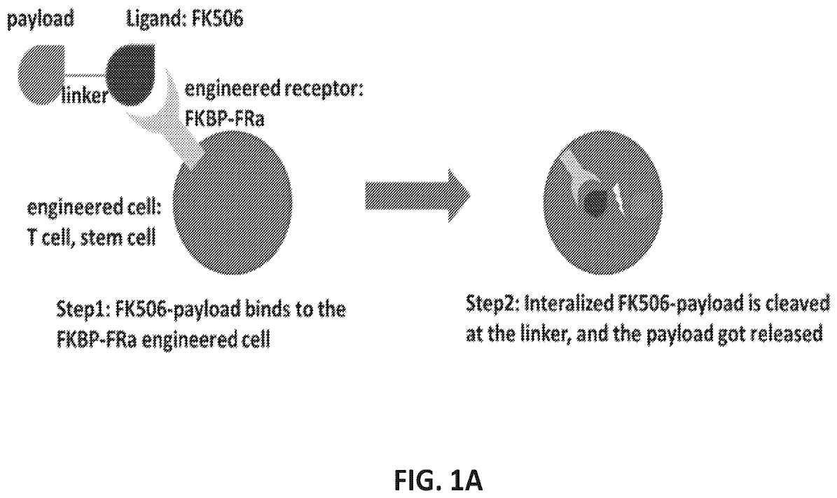

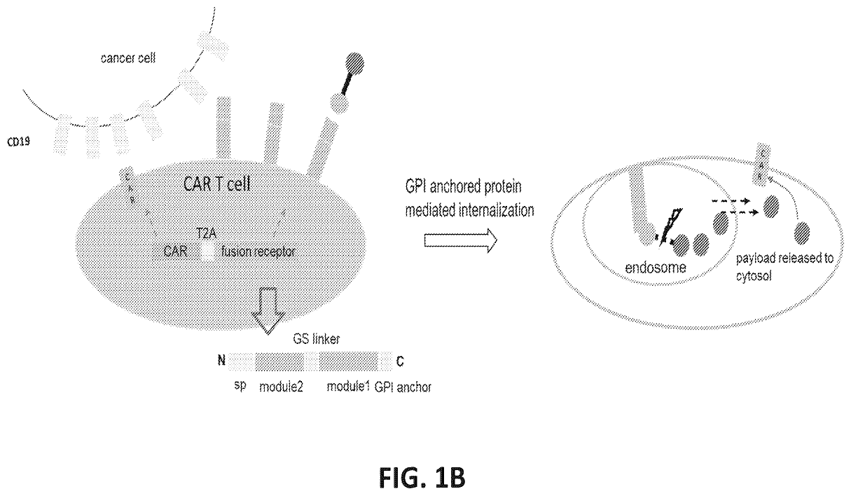

Particularly, in one embodiment, an exemplified pair of small molecule ligand and fusion receptor is chosen as FK506-FKBP. The entire process can be generalized in FIG. 1A, where the FK-506-payload binds to the FKBP-FRa engineered cell first; upon this binding, the transmembrane fusion protein will internalize the payload-linker-FK506. Next, the internalized FK506-payload is cleaved at the linker, and the pay load got released in the cell. Depending on the cell type and the payload type, the released payload drug can exert its desired function.

In FIG. 1B, a specific Chimeric Antigen Receptor T cell mediated cell therapy is illustrated. In this model, a CAR T cell expressing a fusion protein with the structure from N terminus to C terminus comprising a suitable signal peptide, a protein module 2 linked to a protein module 1 of GPI anchor is presented to a cancer cell. In some embodiment, the cancer cell has CD19 surface protein, which will be recognized by the CAR T cell and engaged with the payload associated with the CAR T cell, when a targeting ligand binds to at least one module of the fusion receptor. Typically, with high affinity of the targeting ligand toward any one of these modules, the payload associated with the ligand may be internalized into the target cell and be released to engage the cancer cell through the chimeric antigen receptor.

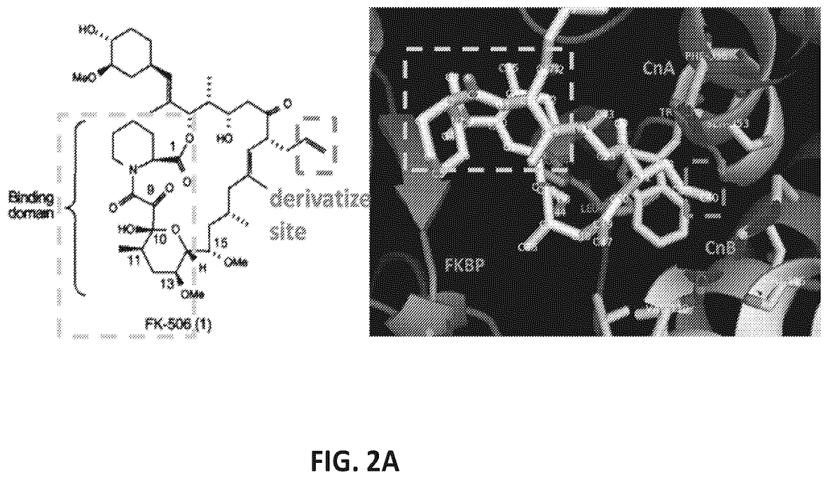

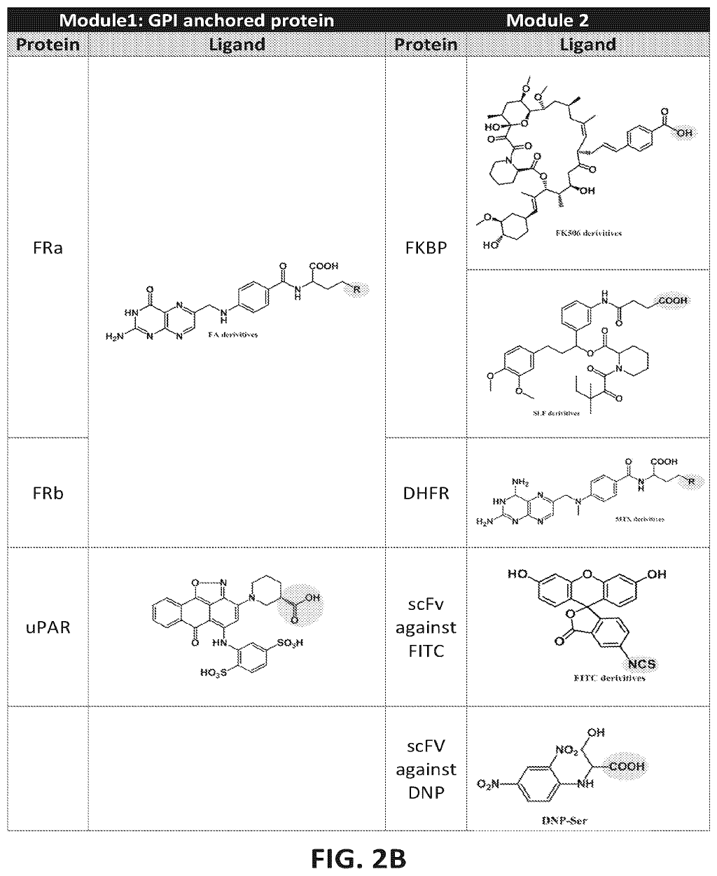

There can be many different combinations of GPI anchored proteins, represented as Module 1 and its ligands, and Module 2 target cell surface protein and its respective ligands, represented in FIG. 2B. It is contemplated that either module 1 protein or module 2 protein, or both can engage a high affinity targeting ligand to facilitate the ligand conjugated payload delivery. For example, it is feasible to have a fusion protein comprising FRa-Linker-FKBP structure, wherein FRa engaging with an FA derivative, at the same time, FKBP engaging a FK506 derivative, either FA derivative or FK 506 derivative or both can be linked to a payload, such as a cytotoxic drug, or an imaging agent, or a modulator. With such flexibility of carrying same or different payload, one can achieve some unexpected, synergy or regulatory effect of different or same payload, or to observe the target cell if the payload is an imaging agent. The advantages of flexibility and diversity of payload delivery of this system will be appreciated with more examples.

Similarly, another embodiment of ligand paired with a GPI anchored protein can be

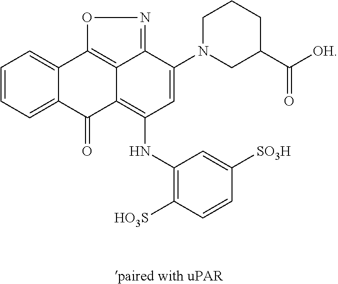 [SMILES: O=C1c2ccccc2-c2onc3c(N4CCCC(C(=O)O)C4)cc(Nc4cc(S(=O)(=O)O)ccc4S(=O)(=O)O)c1c23]

It is also contemplated that FITC or its derivatives may bind to a single chain fragment of variant (scFv) of an antibody against FITC, for example, a ligand structure of

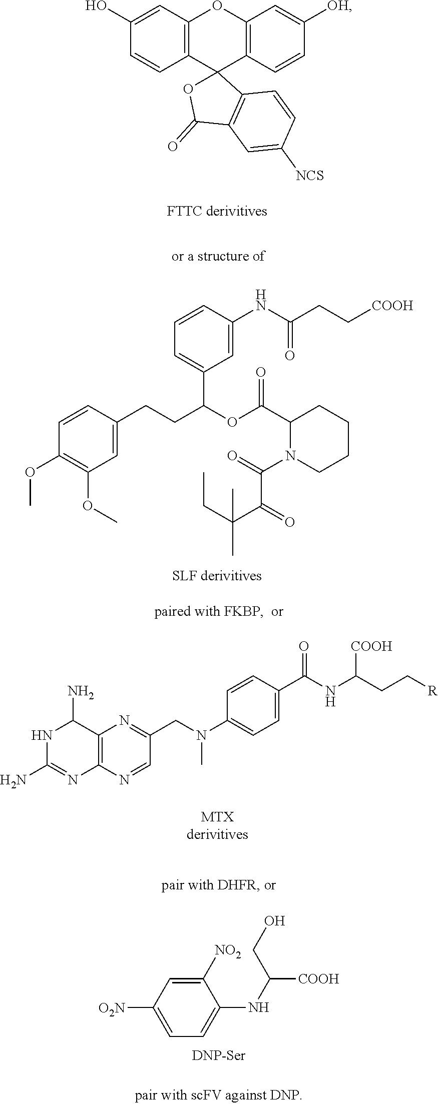

It is worth mentioning a few advantages of choosing FK506-FKBP as an exemplary ligand-protein pair in this delivery system. 1. FKBP is not a membrane protein that naturally present on the mammalian cell membrane, so that the FK506-payload conjugate will specifically bind to the target cell; 2. FKBP protein is a relatively small protein with molecular weight of 12 kDa, which makes it easier to be fused with other receptors with minimum perturbation to the receptors' structure and internalization properties. 3. FK506 is not naturally present inside human being, so that the fusion receptor will not be blocked; 4. The binding affinity between FK506-FKBP is around 4 pM so that the payload drug can be delivered with high affinity; 5. The co-crystal structure of FK506-FKBP is available and the well-established derivative site of FK506 preserves the binding of FK506-FKBP while abolishes the unwanted binding between FK506 and calcineurin (See FIG. 2A).

It is contemplated that the sequence of FKBP can be modified, and the corresponding FK506 ligand can be modified accordingly to the extent that the modified versions of FKBP and modified version of FK506 still have the desired affinity as exemplified herein or better than the current disclosure.

For the other portion of the fusion protein, folate receptor (FR) is chosen for its well understood internalization process as a monomer. Prior research shows that ‘magic carbonate’ linked folic acid conjugate can be internalized through FR and cleaved by the reductive environment inside the cell. Using this mechanism, the FK506-FKBP conjugated drug payload is internalized by FR and will then be released to the cytoplasm.

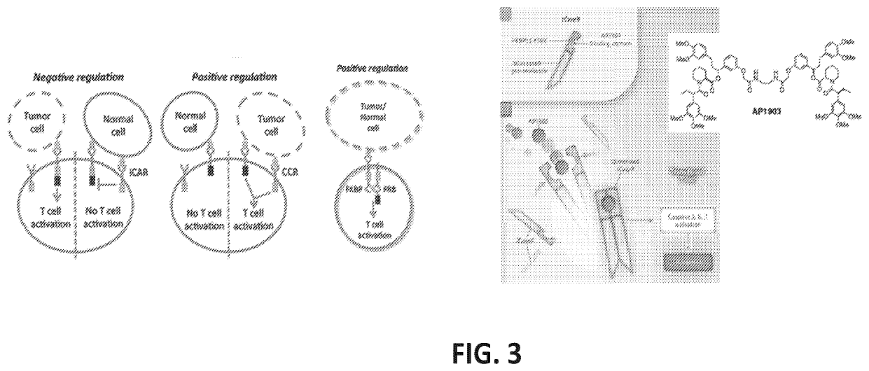

Due to the great potential of CAR T therapy as well as the severe side effects, several controlled CAR T cell designs have been reported. Most of them focused on the ON/OFF switch by incorporating either a Boolean gate or a cascade pathway for the T cell activation (see FIG. 3, left, which depicts negative and positive regulation of the CAR T cell activity, adapted from The quest for spatio-temporal control of CAR T cells, Sun J. etc. 2015). Malcolm K. B. group designed the FKBP-caspase9 fusion protein and use FK506 dimer to induce the apoptosis of the target cell (see FIG. 3, right, which depicts mechanism of AP1903 (FK506 dimer) induced FKBP-caspase9 mediated apoptosis and the structure of AP1903, adapted from Inducible Apoptosis as a Safety Switch for Adoptive Cell Therapy, Malcolm K. B. etc. 2011).

The current disclosure has several advantages over these reported methods: 1. Instead of a binary ON/OFF switch, our platform can delivery multiple kinds of regulating payloads, modifying many aspects of the target cells, thus it has great flexibility compared to binary ON/OFF switch; 2. the controlling moiety, FK506-payload, is a small molecule, which makes it possible for linear control and dosage optimization compared to pre-engineered cells. 3. The platform can be utilized in not only CAR T cell, but many other stem cell based regenerative therapies.

The greatest novelty and most important part of this platform is the multifunctional payloads, which can be selected to either address the potential side effects or improve the efficiency of the cell therapy. For instance, cytotoxic drugs can be delivered to the transplanted cells if: 1. the cells over-proliferate and affect the normal organ or system, like anti CD19 CAR T cell. 2. The cells become tumorigenic, which lies in the lentivirus based gene modification and the intrinsic characteristic of stem cells.

On the other hand, some cell therapies receive less success because of the suppressive microenvironment of the target tissue. For instance, in CAR T cell therapy against solid tumor, aside from the low penetration rate, the proliferation and activation of CAR T is highly suppressed by the MDSC and the tumor cells. This can be potentially alleviated by the RORrt agonist or MAP kinase inhibitor induced T cell activation as well as TLR8 agonist induced expression of granzyme B in CAR T cells. Although intracellular targets, like RORrt, may be more suitable for our payload, membrane receptors such as TLR8 may also be accessible due to the proximity on the cell membrane.

In stem cell regenerative therapy, the payload is more diverse according to the disease models. Instead of delivering a pre-fixed gene, which has been developed by using stem cell as a gene delivery platform, the current disclosure provides a fine tune to transplanted cells and their microenvironment, and obtains the desired phenotype through diverse small molecule payloads. Since the small molecule is conjugated to FK506, which specifically target the FKBP-FRa overexpressed transplant cell, the non-specific targeting of normal tissues of the small molecule is also avoided.

One of the many examples is to induce the overexpression of BMP2 in mesenchymal stem cell (MSC) for bone fracture repair in skeletal regenerative therapy. Meanwhile, the expression level of BMP2 and/or VEGF can be increased by introducing GSK3 beta inhibitor to the transplanted cells through this drug payload delivery system. GSK3 beta inhibitor is a desired drug for bone fracture repair. Therefore, GSK3 beta inhibitors is ideal as a potential payload for further modulating the function of the transplanted MSC as well as the microenvironment within the bone fracture sites.

Another example of this drug payload delivery system goes to the neurodegenerative disease, including Alzheimer, Parkinson disease and etc., where MSC based therapy holds a promising future. MSC has been modified to overexpress GDNF, VEGF and many other cytokines to promote the neuronal regeneration. Meanwhile, small molecules like MAO-B inhibitors has been confirmed to increase the expression of GDNF, NGF and BDNF in astrocytes. Several kinase inhibitors have also been proposed for the treatment of Alzheimer disease, such like PI3K inhibitor (BEZ235), Cdk5 inhibitor (roscovitine) and GSK3b inhibitor (NP-12). Using FK506-FKBP pair targeted specific delivery of these small molecules to MSC in neurodegenerative models will improve the regeneration efficacy while avoiding the side effects of these potent inhibitors and agonists in other non-targeted tissues.

### Material and Methods

### Compounds and Synthesis Procedure

Targeting ligand is linked with payload through linkers. The linker optimization options are listed below, payloads are characterized into 3 classes: I imaging, II cytotoxic drug, III regulatory small molecule drug.

### Linker Optimization

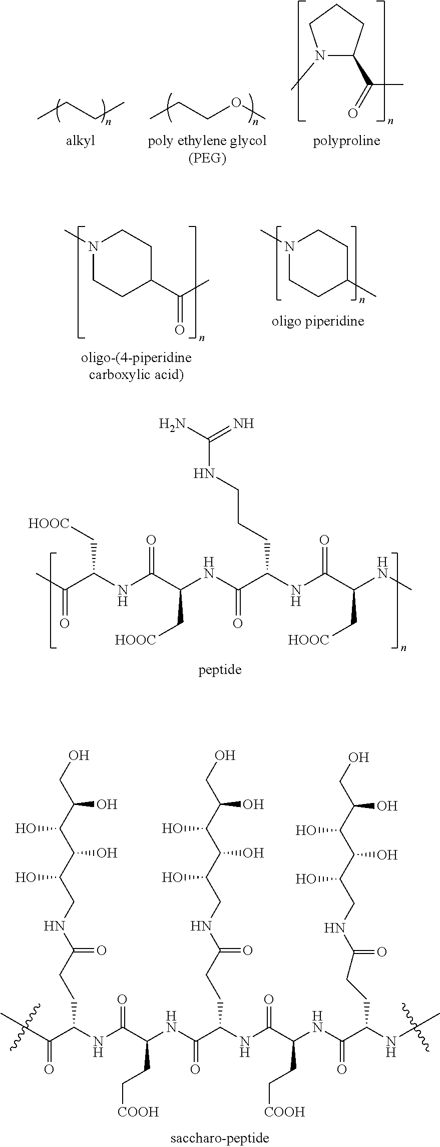

### Compounds Classification

| Class | function | example |
|---|---|---|
| I | Imaging | Fluorescent dye: Rhodamine, FITC Radioisotope |
| | | imaging: EC20 chelating head, NOTA, DOTA |
| II | Cytotoxic | Anti-microtubule drug: Tubulysin, DM1, DM4 |
| | drug | |
| III | Modulator | Kinase inhibitor: Dasatinib, MEK 1/2i, PI3Ki |
| | | siRNA: mi181a1 |

### Detailed Compound Structure and Synthesis Route

### FK506-Rhodamine

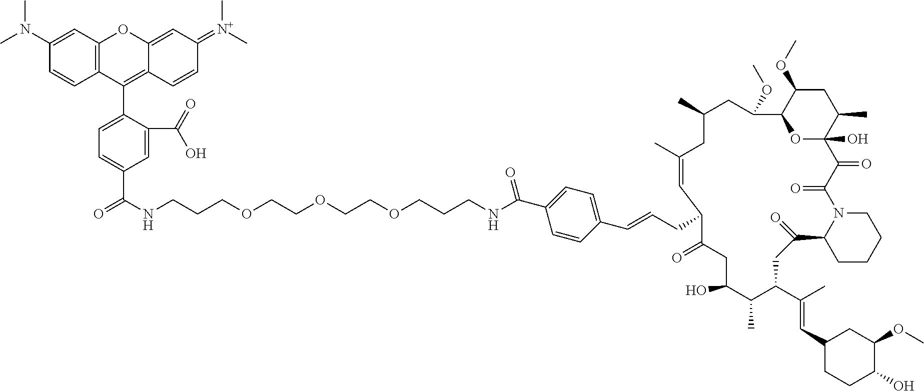

Procedure: Rhodamine-NHS ester (1.0 equiv.) in dimethylformamide was reacted with Boc-NH-PEG₃-NH₂ (1.2 equiv.) and diisopropylethylamine (3.0 equiv.) for 2 h at room temperature. The product was purified by preparative reverse-phase HPLC with a UV detector. The purified Rhodamine-PEG₃-NH-Boc conjugate (1.0 equiv.) was subjected to Boc deprotection by stirring in a 1:10 TFA-dichloromethane system for 2 h. The crude free amine product was then dissolved in dimethylformamide and activated with EDC (2.0 equiv.), HOBT (2.0 equiv.) in the presence of diisopropylethylamine (3.0 equiv.). After 15 minutes, FK506-CO₂H (1.2 equiv., synthesized using the procedure in the reference: Bioorg. Med. Chem., 17 (2009) 5763-5768) was added and the reaction mixture was stirred overnight. The final FK506-Rhodamine conjugate was isolated after purification on preparative reverse-phase HPLC with a UV detector (monitored at wavelength of 280 nm). The crude product was loaded onto an Xterra RP18 preparative HPLC column (Waters) and eluted with gradient conditions starting with 95% 5 mM sodium phosphate (mobile phase A, pH 7.4) and 5% acetonitrile (mobile phase B) and reaching 0% A and 50% B in 35 min at a flow rate of 12 mL/min. Retention time of the product peak=2.5 min during the gradient (0-50% JB) in a 7 min analytical HPLC-MS analysis. ESI m/z=1539.6. Abbreviations: PEG =polyethylene glycol; EDC=1-Ethyl-3-(3-dimethylaminopropyl)carbodiimide; HOBT=Hydroxybenzotriazole; HPLC=High Performance Liquid Chromatography.

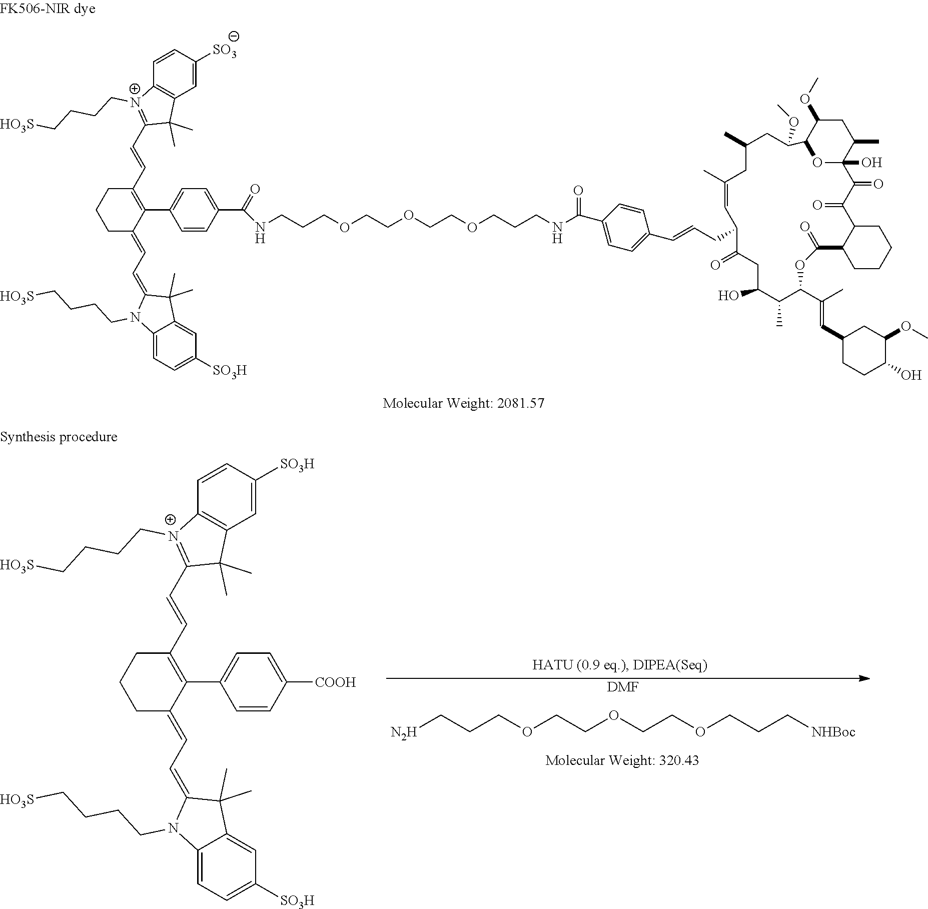

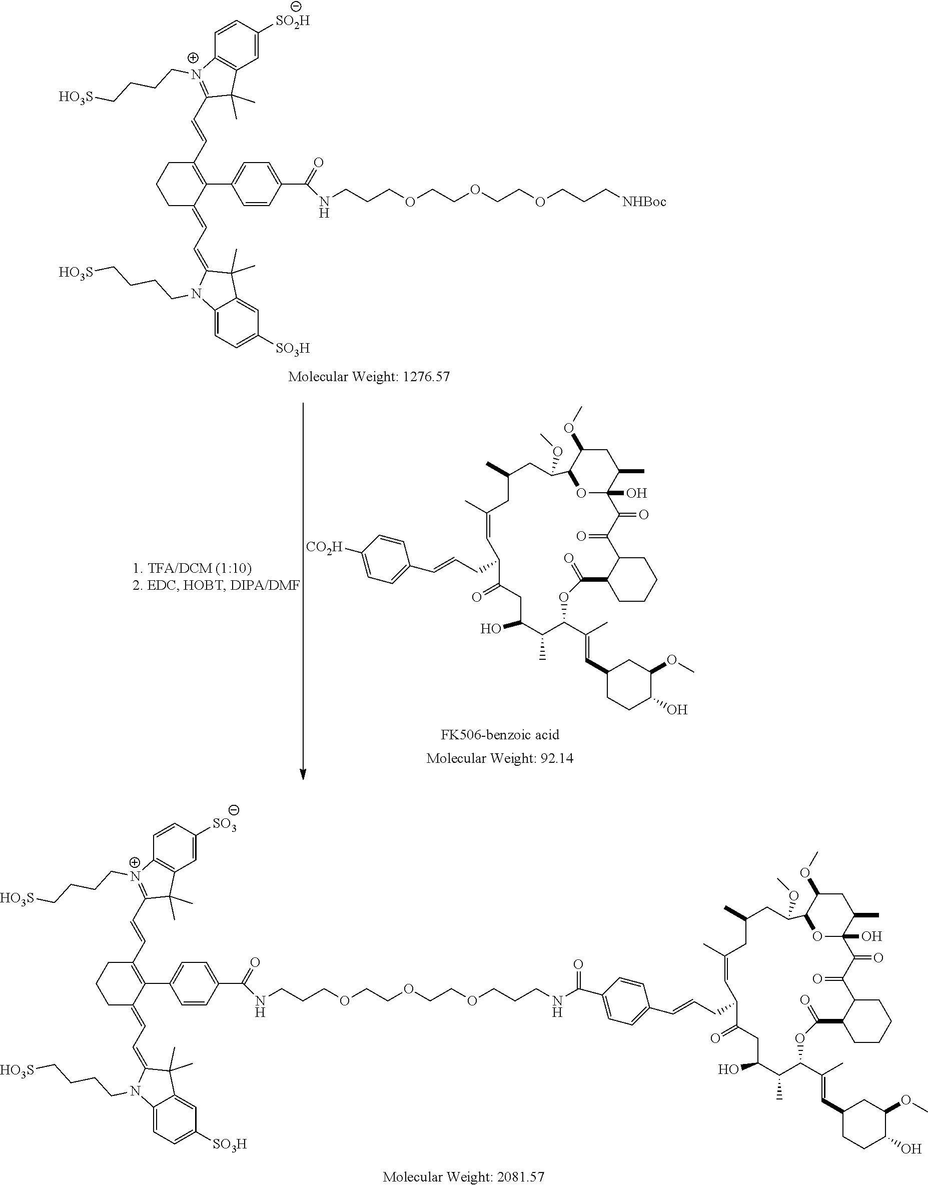

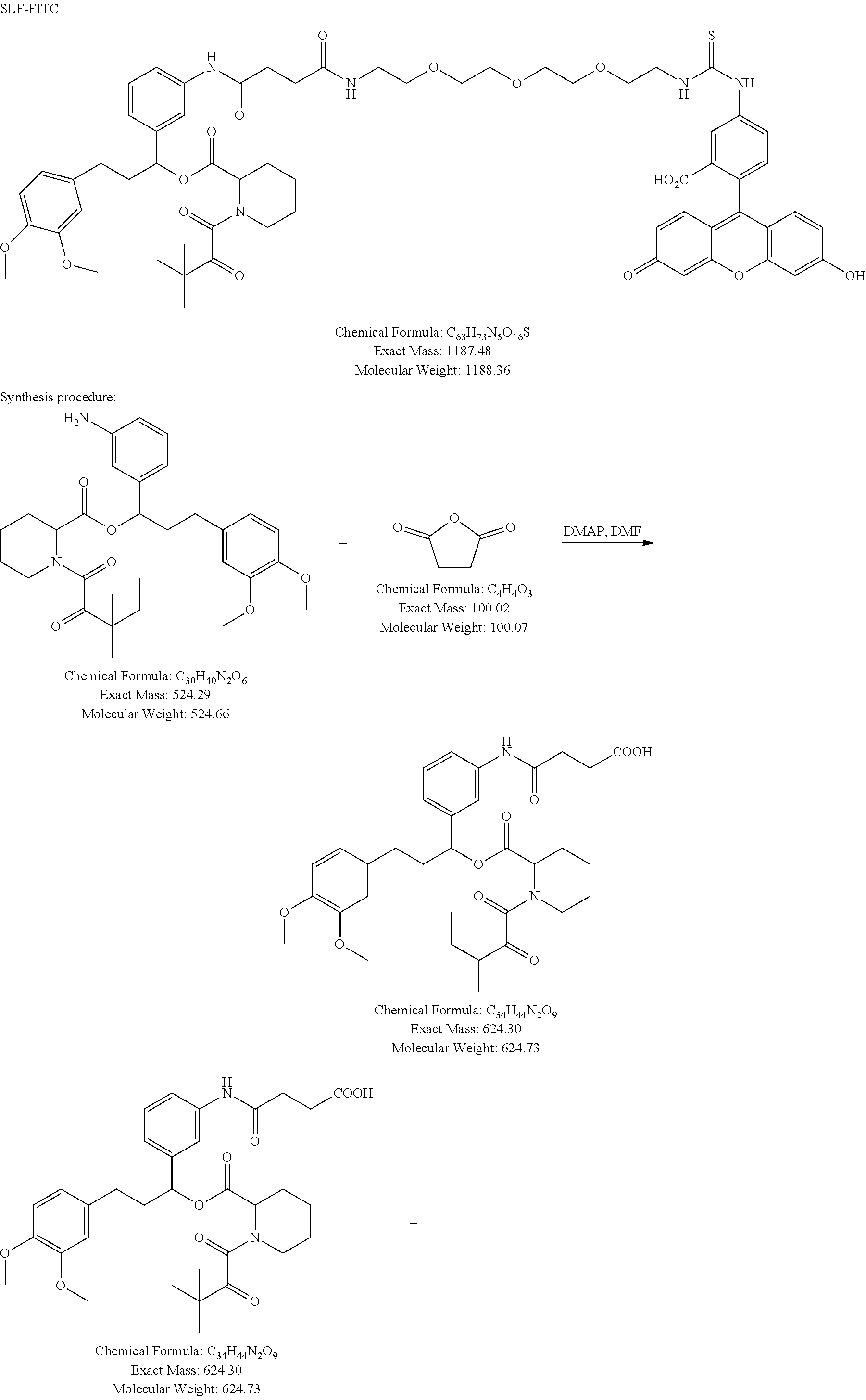 [SMILES: COc1ccc(CCC(OC(=O)C2CCCCN2C(=O)C(=O)C(C)(C)C)c2cccc(NC(=O)CCC(=O)NCCOCCOCCOCCNC(=S)Nc3ccc(-c4c5ccc(=O)cc-5oc5cc(O)ccc45)c(C(=O)O)c3)c2)cc1OC ; CCC(C)(C)C(=O)C(=O)N1CCCCC1C(=O)OC(CCc1ccc(OC)c(OC)c1)c1cccc(N)c1 ; O=C1CCC(=O)O1 ; CCC(C)C(=O)C(=O)N1CCCCC1C(=O)OC(CCc1ccc(OC)c(OC)c1)c1cccc(NC(=O)CCC(=O)O)c1 ; COc1ccc(CCC(OC(=O)C2CCCCN2C(=O)C(=O)C(C)(C)C)c2cccc(NC(=O)CCC(=O)O)c2)cc1OC]

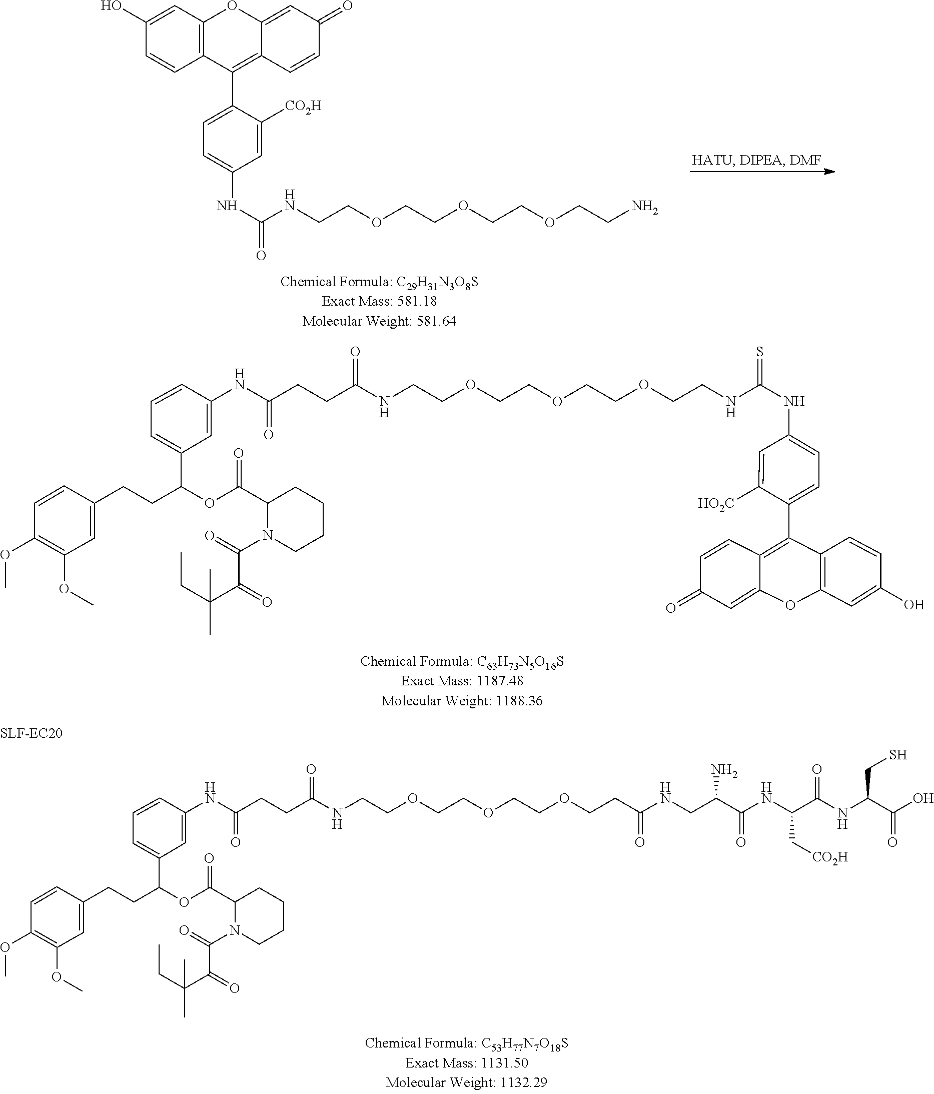 [SMILES: NCCOCCOCCOCCNC(=O)Nc1ccc(-c2c3ccc(=O)cc-3oc3cc(O)ccc23)c(C(=O)O)c1 ; CCC(C)(C)C(=O)C(=O)N1CCCCC1C(=O)OC(CCc1ccc(OC)c(OC)c1)c1cccc(NC(=O)CCC(=O)NCCOCCOCCOCCNC(=S)Nc2ccc(-c3c4ccc(=O)cc-4oc4cc(O)ccc34)c(C(=O)O)c2)c1 ; CCC(C)(C)C(=O)C(=O)N1CCCCC1C(=O)OC(CCc1ccc(OC)c(OC)c1)c1cccc(NC(=O)CCC(=O)NCCOCCOCCOCCC(=O)NC[C@H](N)C(=O)N[C@@H](CC(=O)O)C(=O)N[C@@H](CS)C(=O)O)c1]

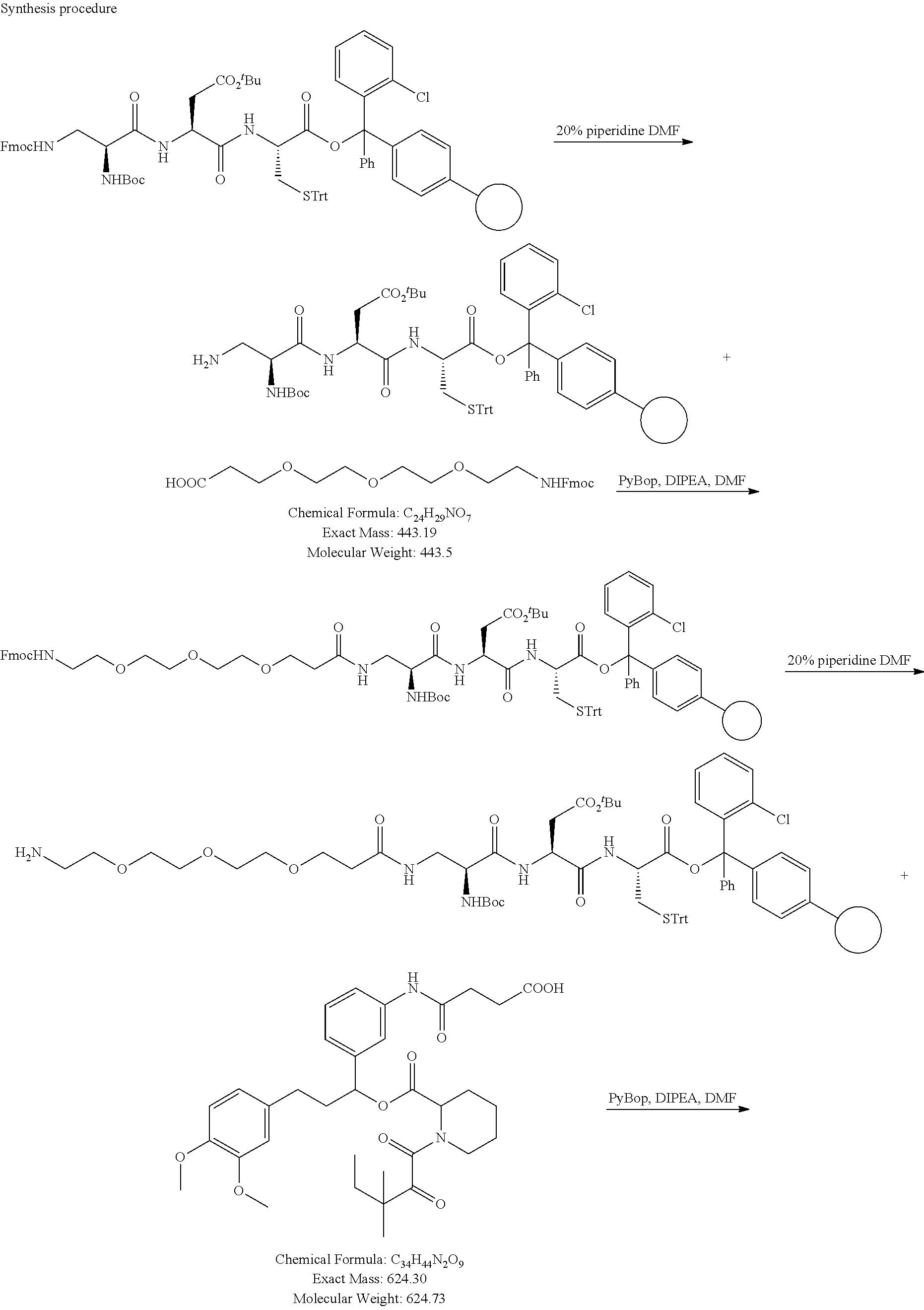

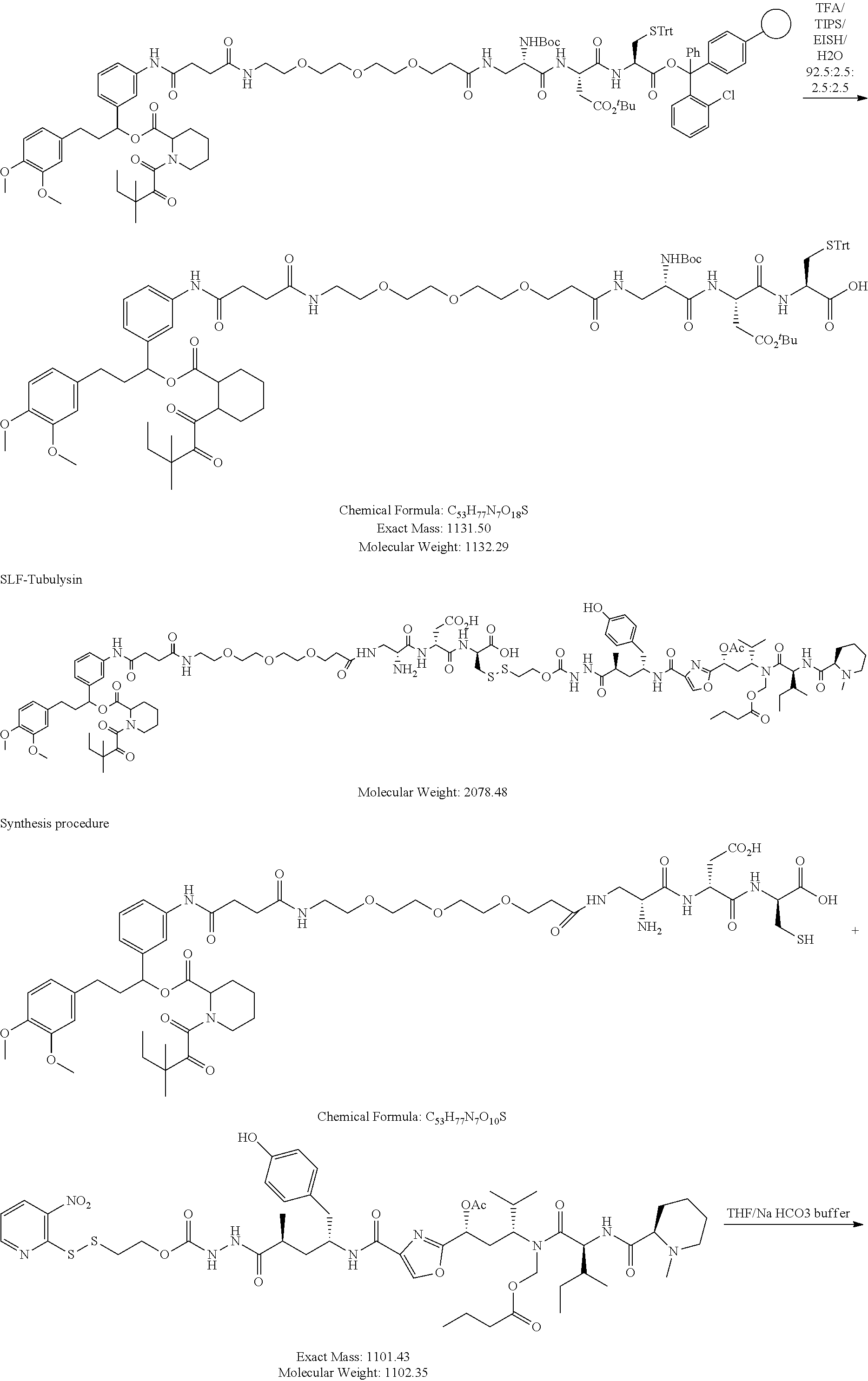

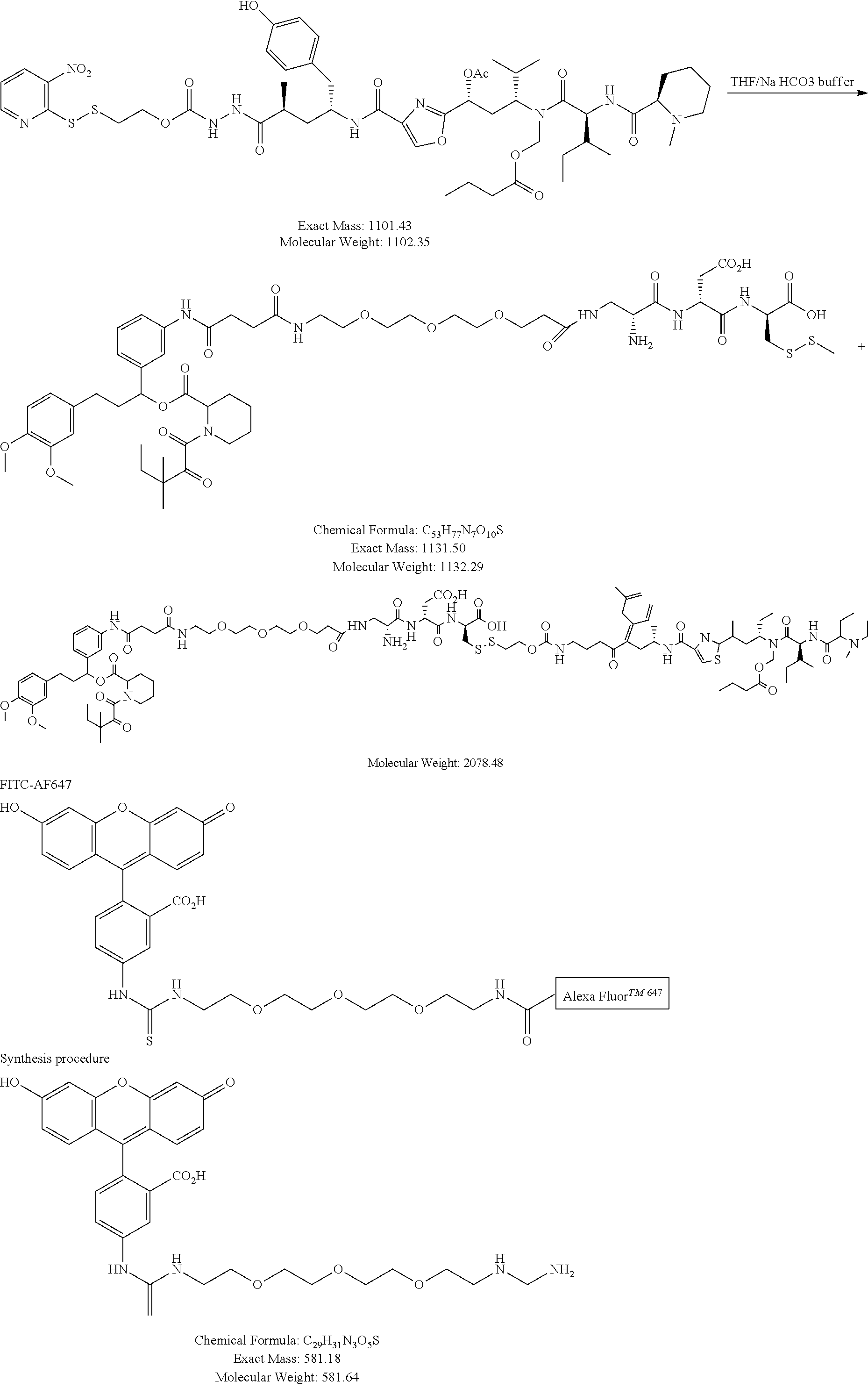

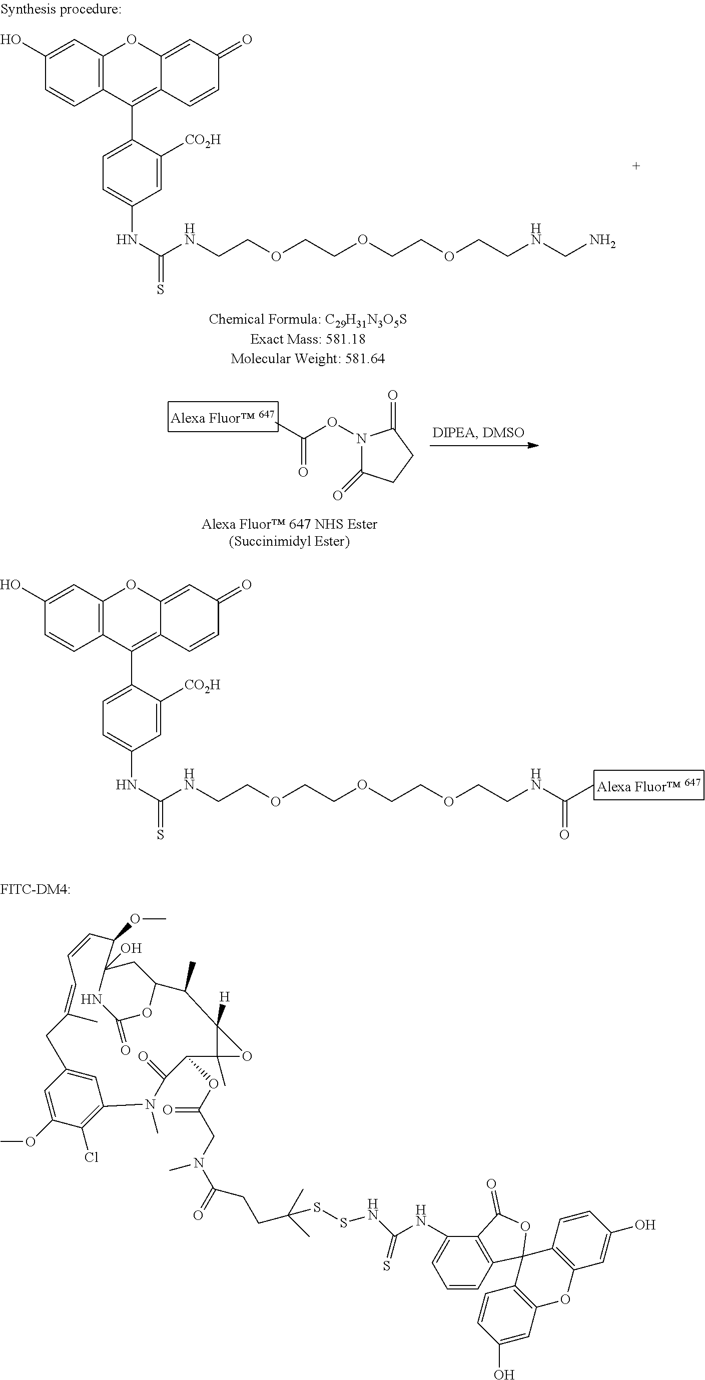

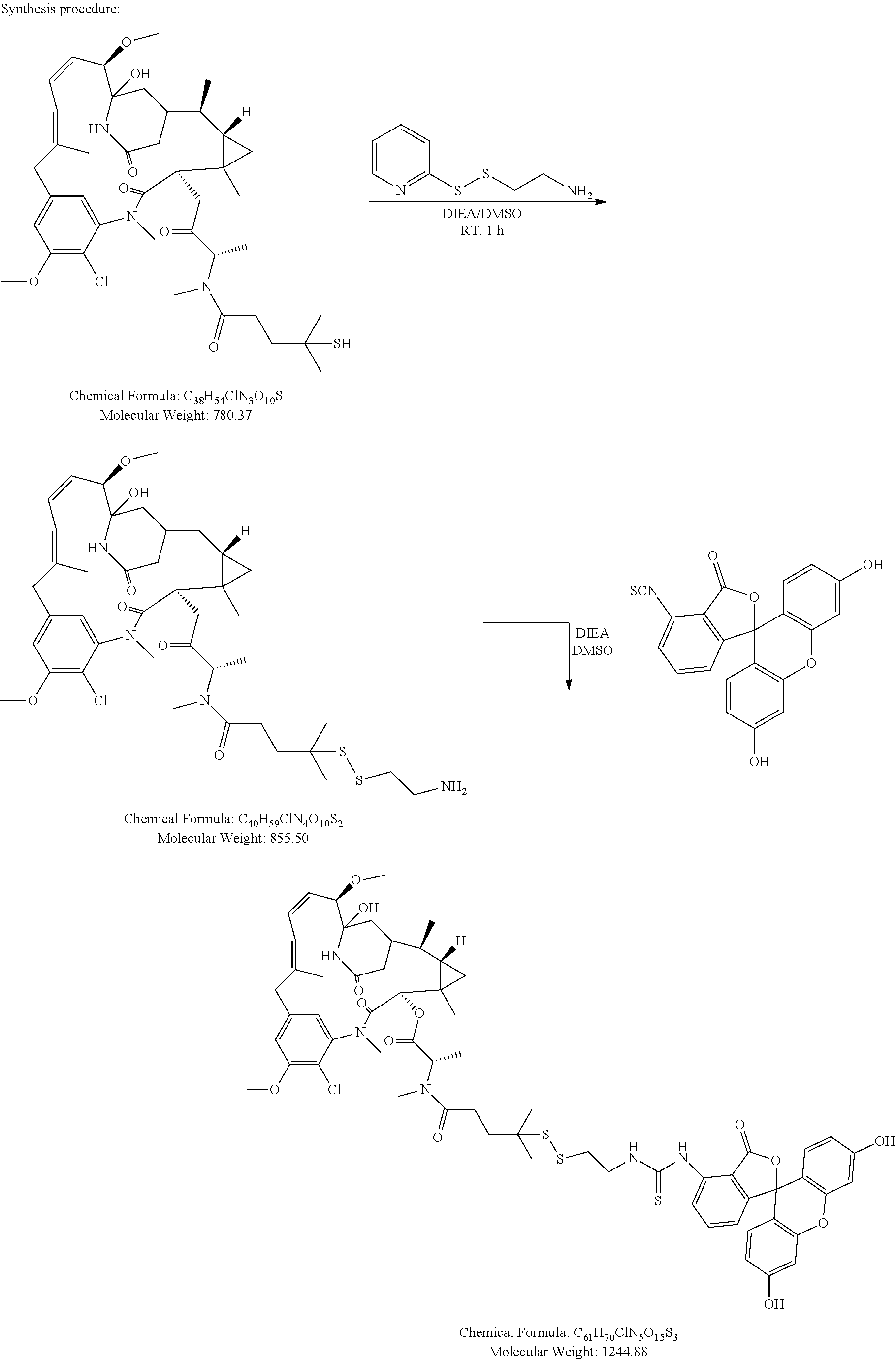

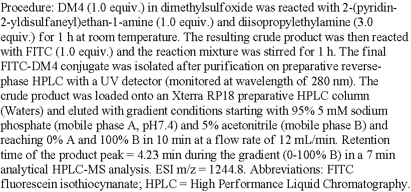

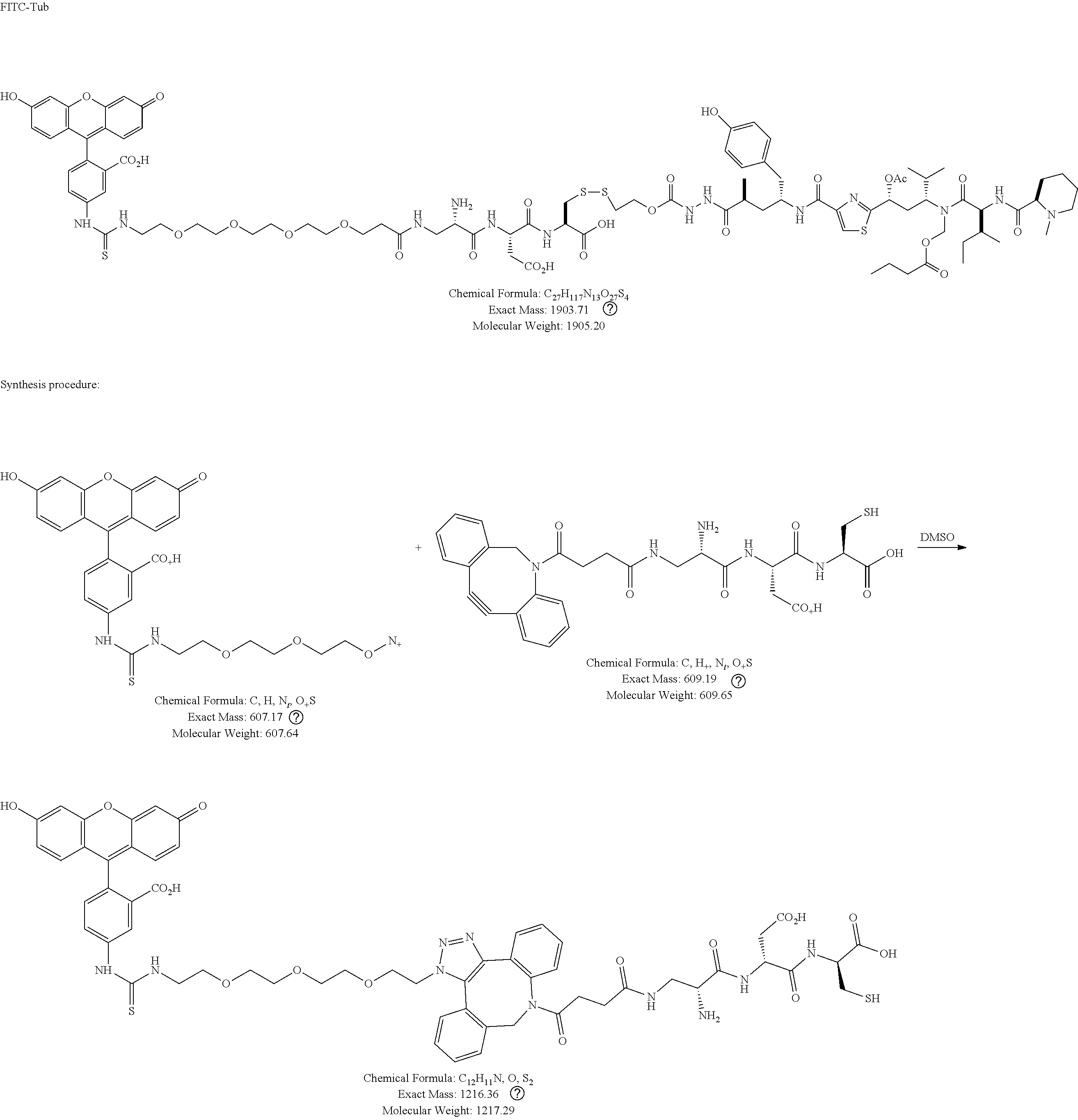

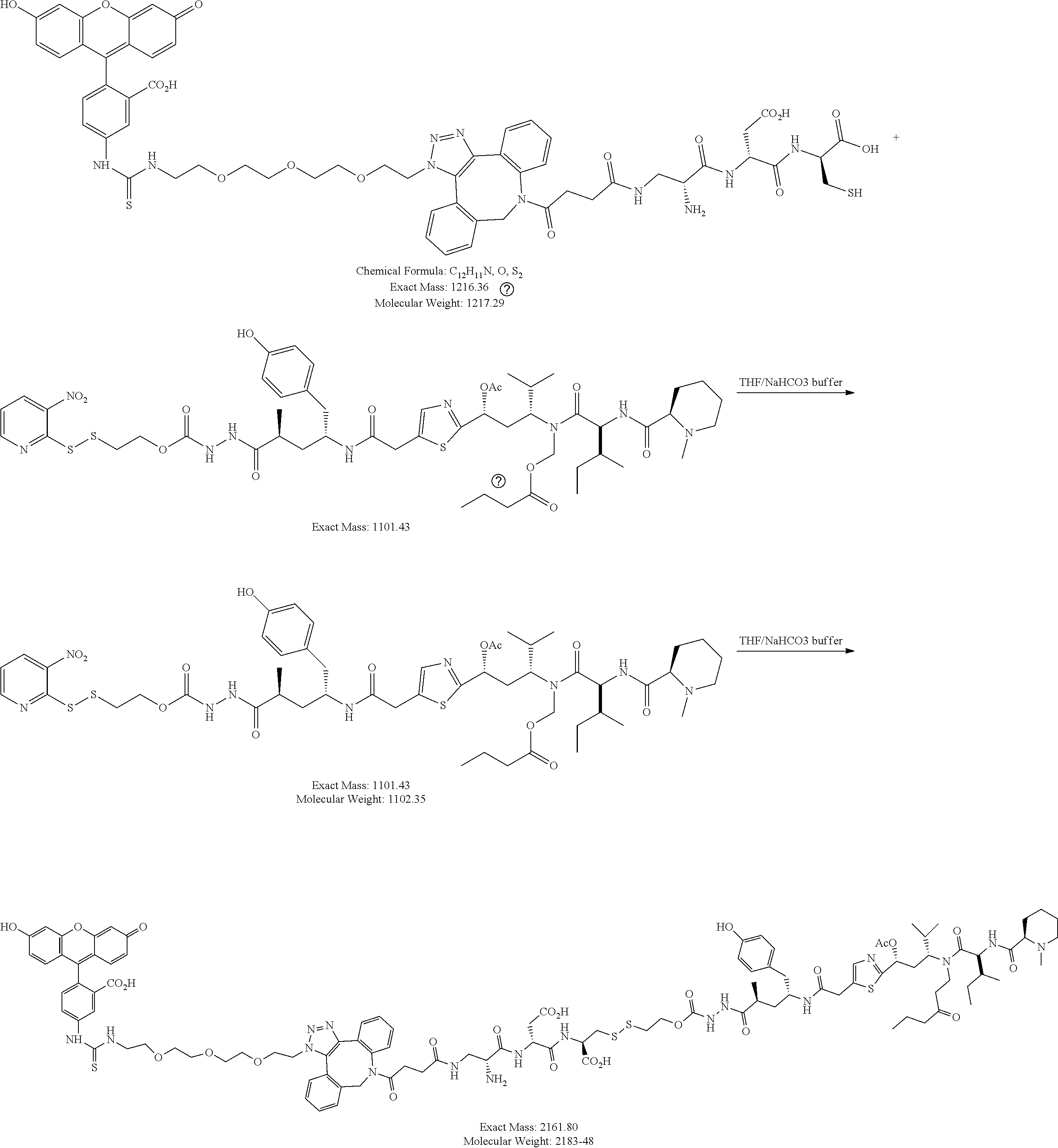

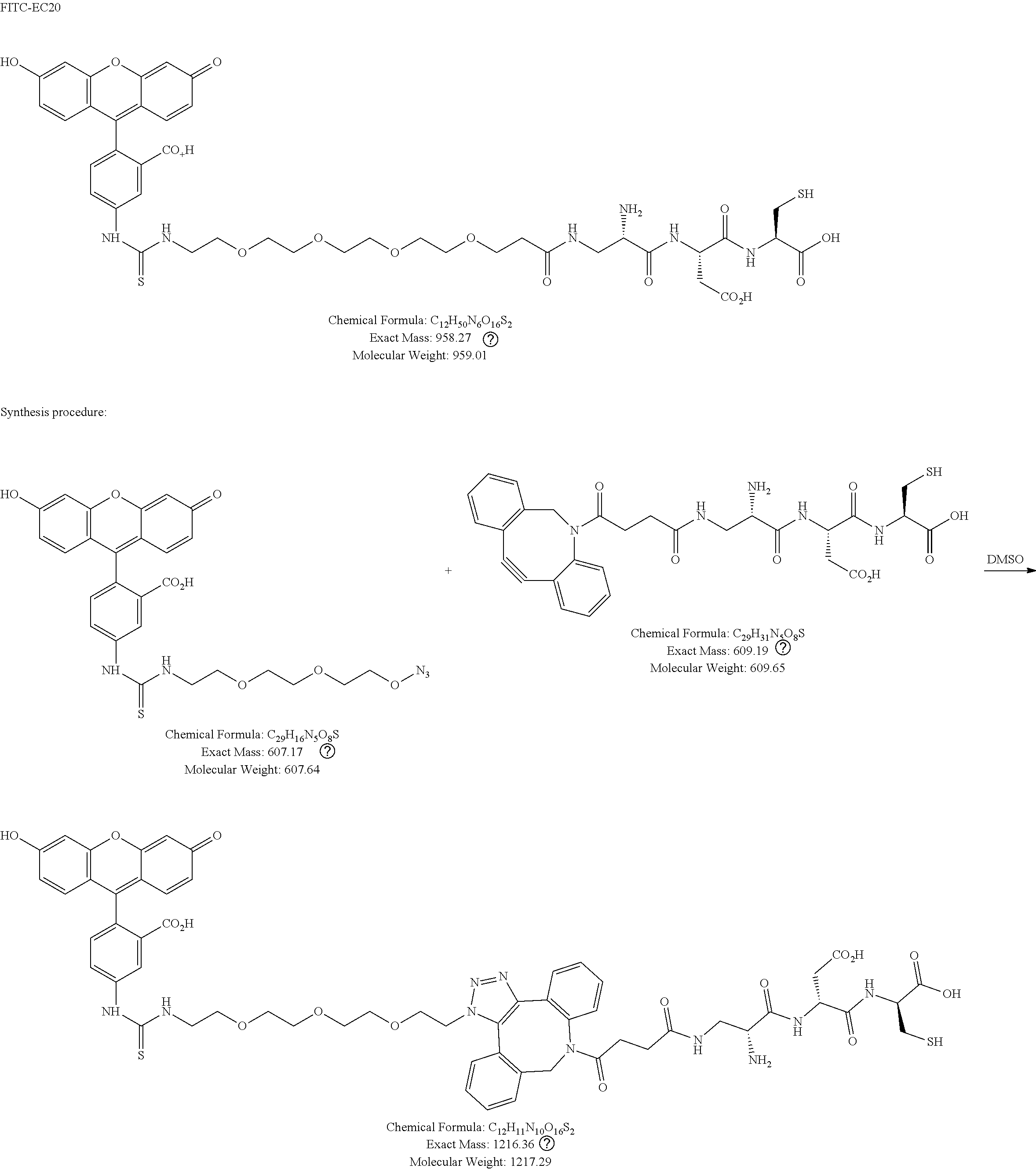

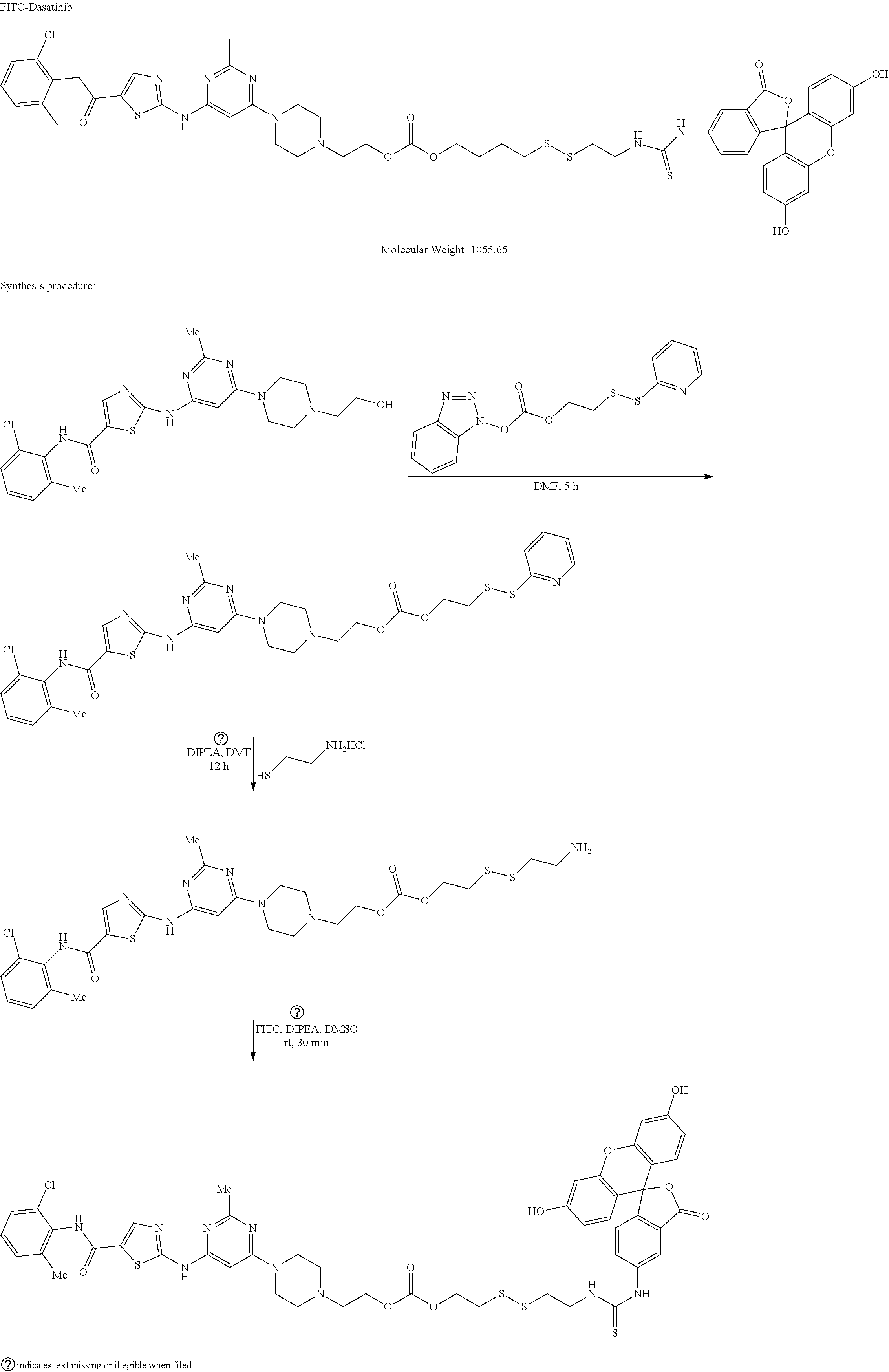

### The Experiment Procedure

### Cell Culture

293TN cells were cultured in DMEM with 10% FBS, no antibiotic for lentivirus packaging. Raji and Jurkat cells were cultured in RPMI-1640 with 10% FBS, 10% Penicillin/Streptomycin. Primary human T cells were isolated from hPBMC using ficoll, enriched by negative selection with EasySep™ Human T Cell Enrichment Kit (19051, Stemcell Tech), activated by Dynabeads CD3/CD28 (11161D, Thermo Fisher) for 1 days, cultured with TexMACS medium supplemented with 30IU hIL2 (130-097-745, Miltenyi Biotec Inc.). T cells were cryopreserved in RPMI-1640 with 20% human AB serum (HP1022, Valley Biomedical) and 10% DMSO.

### Lentivirus Packaging

Pantropic VSV-G pseudotyped lentivirus was produced via transfection of 293TN cell with the transgene expression vector and packaging plasmid mix (CPCP-K2A, Cellecta) using Lipofectamine 2000. At 24 h, viral supernatant was harvested, concentrated and then added to certain cell lines or the primary T cells that were thawed the same day. For T cell transduction, cells were centrifuged at 2500 rpm for 90 mins, 37 degree after adding the virus supernatant and 8 ug/ml polybrene.

### Binding Assay

For binding assay, cells were incubated with ligand-dye alone or with ligand-dye and free ligands (100×, preincubated for 30 min) for 30 min, at 4 degree. Cells were washed 3 times after incubation, and re-suspended in 2% FBS PBS, 7-AAD were added to gate out the dead cells. FRET imaging of FK506-Rhodamine and FA-S0456: To understand the occupation of the fusion receptor, FKBP-FRa+ jurkat cells are incubated with FA/FA-S0456, FK506/FK506-Rhodamine at the indicated sequence and concentration. FRET is visualized by the loss of intensity of FA-Rhodamine as its energy transferred to FA-OTL38 on the same or nearby receptor, detected by BD Fortessa flow cytometer. Results were analyzed using Flow Jo software.

### PI-PLC Treatment to Release the GPI Anchored Protein

1×10⁵ cells were incubated with 5 mU or 50 mU PI-PLC (P5542-5UN, Sigma) in digestion buffer (2% BSA) at 37 degree for 30 min; after incubation, cells were washed three times by PBS and then incubated with ligand-dye for 30 min on ice.

### Cell Viability Assay

Cells were seeded to 96 well plate and incubated with different concentration of certain ligand-cytotoxic drug for 2 h, with or without 100× preincubation of the free ligand competition. After 2 h incubation, cells were washed by warm medium 3 times and replenished with fresh medium. After 72 h, cell number were tested by CellTiter-Glo® assay (G7570, Promega) or quantified by ligand-dye staining of receptor positive cell.

### CAR T cell lysis effect

1×10⁵ CAR T and certain number of Raji cell were co-incubated in 96 well plate, according to the E:T ratio, with or without the treatment drug. After 24 h, 100 ul supernatant were taken out for LDH assay. Lysis percentage were calculated as (treatment group—CAR T only)/(maximum lysis—CAR T only)%

### Exhaustion of CAR T Cell

1×10⁶ Raji cell were repeated added to 1×10⁶ CAR T cell in 24 well plate every 12 h, without changing the medium. Exhaustion status were characterized by lower lysis effect and higher expression of co-inhibitory molecules: PD-1, LAG-3 and Tim-3.

### In Vivo Ablation of the Fusion Receptor Positive CAR T by Ligand-Cytotoxic Drug

4×10⁵ luc+Raji cells were iv injected into the NSG mice. After 6 days, 1×10⁷ fusion receptor positive CAR T cells were iv injected. On Day 8, ligand-cytotoxic drug (0.5 umole/kg, 1 umole/kg) were iv injected once. IL2, INFr were measured by ELISA using serum sample taken every 3 days after the CAR T injection. CAR T cell in peripheral blood were counted by flow cytometry.

### In Vivo Modulation of Fusion Receptor Positive CAR T by Ligand-Drug

4×10⁵ luc+Raji cells were iv injected into the NSG mice. After 6 days, 1×10⁷ fusion receptor positive CAR T cells were iv injected. On Day 7, ligand-drug drug (0.5 umole/kg, 1 umole/kg) were iv injected once. In the case of CAR T exhaustion model, 1×10⁵ CAR T were iv injection on Day 6, once the CAR T population were shown in peripheral blood and tumor burden is not stabilized and continue to increase, ligand-drug (0.5 umole/kg, 1 umole/kg) were iv injected.

### In Vivo Imaging and Tracking of Fusion Receptor Positive CAR T

2×10⁶ Raji were subcutaneously injected to the right should of NSG mice. 14 days later, 1×10⁷ fusion receptor positive CAR T were iv injected. On Day 16, animals were administrated 99mTc-bound conjugates (10 nmol, 150 μCi) by iv injection and imaged by SPECT imaging machine.

### EXAMPLES

### 1. Design of Fusion Protein and Its Expression

### 1.1 Design of FKBP-FR Fusion Receptor (SEQ ID NO:2)

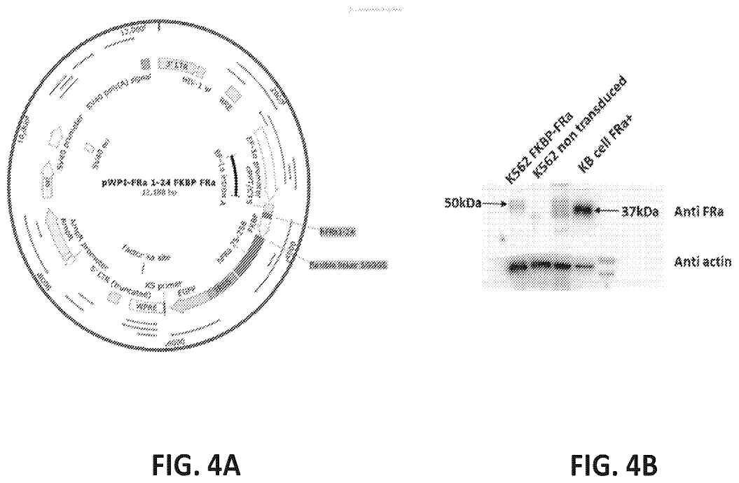

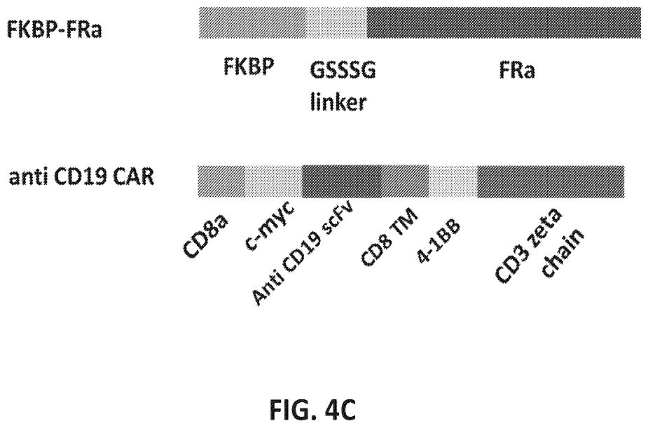

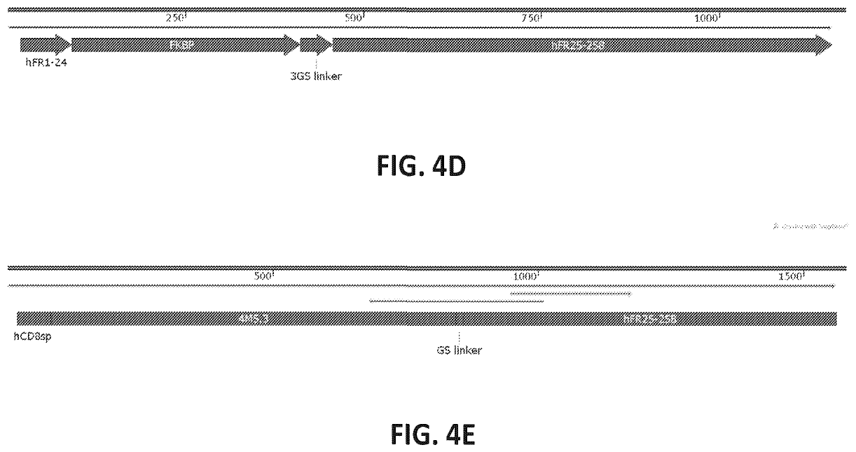

Synthesis of FK506 derivatives: FK506-Rhodamine and FK506-tubulysin Synthesis was described in materials and methods section. hFRa is a GPI anchored membrane protein, which has 24 amino acids on the N terminal as a signal peptide. In order to preserve the membrane presentation and internalization property, we choose to use the full length of FRa, and incorporate the hFKBP sequence as well as a flexible peptide linker (SGGGS) between T24 and R25 of hFRa (FIG. 4a). The flexible linker is chosen to be resistant to common enzyme digestions in human body. The whole sequence is then inserted into a pWPI lentivirus expression vector, with EF1a as the desired promoter for protein expression in transduced T cell.

### 1.2 Expression of FKBP-FR Fusion Receptor in Transduced Cells

Expression of FKBP-FRa is confirmed by western blot in lentivirus transduced K562. Transduced K562 cell lysis shows specific band around 50 kDa compared to non-transduced cells against hFRa antibody (FIG. 4b).

### 1.3 Construction of Fusion Protein of FKBPFR3GS (Noted as FF3)

See FIG. 4d (SEQ ID NO: 12). From N terminal to C terminal it has 1-24 aa of human FRa as the signal peptide, human FKBP protein, three Gly-Ser linker and then 2-258 aa of human FRa. In a construct design of FKBPFR1GS (noted as FF1), the three Gly-Ser linker of FF3 is substituted with one Gly-Ser linker with other parts unchanged. As will be shown in the binding assays, increasing linker length reduces the interference between the two components in the fusion protein.

### 1.4 Construction of Fusion Protein of FITC-svFv-FR With GS Linker

See FIG. 4e. The construct is also named as 4M5.3 FR (SEQ ID NO: 13). From N-terminal to C-terminal, it has hCD8 signal peptide, svFv of 4M5.3 (against FITC), GS linker, 25-258 aa of human FRa.

### 1.5. In Vivo Noninvasive Tracking of FKBP-FRa/FKBPtFRa Positive Cells by FK506-99mTc PET Imaging

For CAR T cell model, 1×10⁶ KB cells are subcutaneously implanted in NSG (Jackson laboratory). After the tumor reaches 100 mm³, 15 million anti-FITC CAR+ FKBP-FRa+ or anti-FITC CAR+ FKBP-FRa− human T cells are intravenously injected to the mice. FITC-FA is injected at the indicated days to induce the proliferation of the CAR T. Mice are imaged every two days after the CAR T implantation by the following procedure. At the day of imaging, FK506-EC20 head is formulated with 99mTc according to previous report. 200 uCi 99mTc in 100 ul solution is i.v. injected to each mouse and whole body image is taken by MiLab PET/CT, focusing on the tumor area, spleen and lymph nodes. 3D images are reconstructed by PMOD software. After the last imaging at around day 10 after CAR T implantation, mice are euthanized and 99mTc distribution in each organ are counted by gamma counter.

For Hematopoietic Stem Cell transplantation model, humanized NSG mice are generated as reported before. 10 million CD34+ FKBP-FRa+ hHSC are i. v infused into the humanized NSG mice. 4 months later, whole body image is taken using FK506-99mTc as mentioned above, focusing on the bone marrow and spine.

### 2. FKBP-FRa Fusion Receptor Specifically Binds and Internalizes FK506-Payload

### 2.1 FKBP-FRa Fusion Receptor Specifically Binds FK506-Rhodamine

For binding assay, cells were incubated with ligand-dye alone or with ligand-dye and free ligands (100×, preincubated for 30 min) for 30 min, at 4 degree. Cells were washed 3 times after incubation, and re-suspended in 2% FBS PBS, 7-AAD were added to gate out the dead cells. BD Fortessa flow cytometer were used. Results were analyzed using FlowJo software.

### 2.2. Binding of FK506-Rhodamine by FKBP-FRa Fusion Receptor (FRET Imaging of FK506-Rhodamine and FA-S0456)

To understand the occupation of the fusion receptor, FKBP-FRa+ jurkat cells are incubated with FA/FA-S0456, FK506/FK506-Rhodamine at the indicated sequence and concentration. FRET is visualized by the loss of intensity of FA-Rhodamine as its energy transferred to FA-OTL38 on the same or nearby receptor, detected by BD Fortessa flow cytometer.

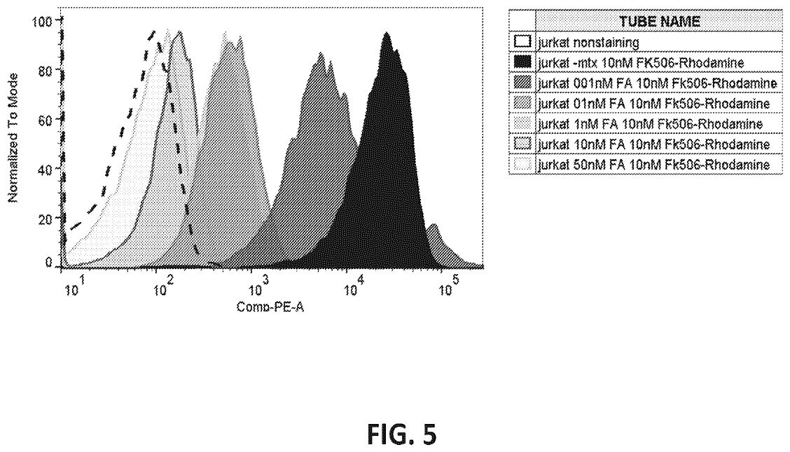

FIG. 5 shows binding of Folate acid in FKBPFR1GS fusion protein blocks the binding of FK506-Rhodamine at as low as 0.01 nM and totally abolishes FK506 Rhodamine binding at 50 nM.

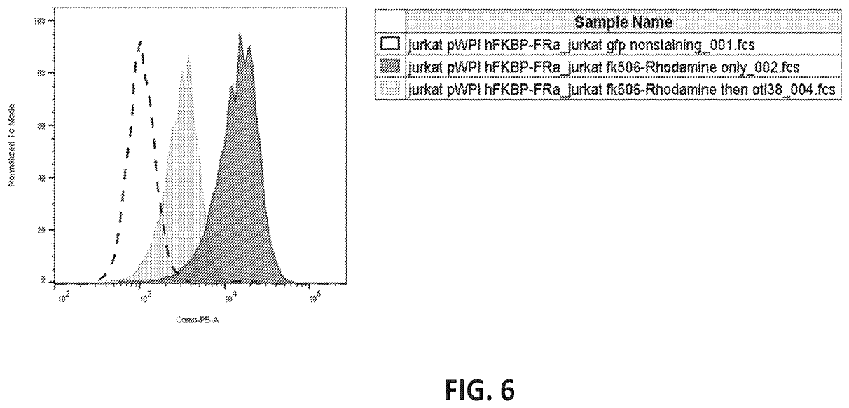

FIG. 6 shows FKBPFR1GS jurkat cells have decreased FK506-Rhodamine intensity after binding to OTL38, a folate receptor targeted dye. FRET from FK506-Rhodamine (donor) to OTL38 (FA-S0456, acceptor, ex/em: 774/794 nm) indicates the interaction between FR and FKBP within the fusion receptor.

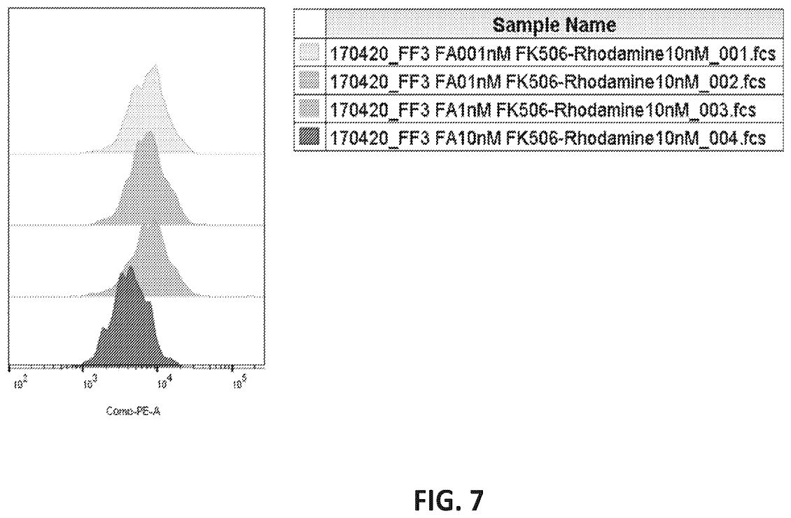

FIG. 7 shows increasing the linker length between FKBP and FR significantly lowers the interference between the two components of the fusion protein. Compare to FF1 (1GS between FKBP and FR), FF3 (3GS between FKBP and FR) preserves the binding of FK506-Rhodamine in the presence of 10 nM FA, which is comparable to the physiological concentration of FA in human body.

### 2.3. Release of GPI Anchored FF3 Fusion Receptor

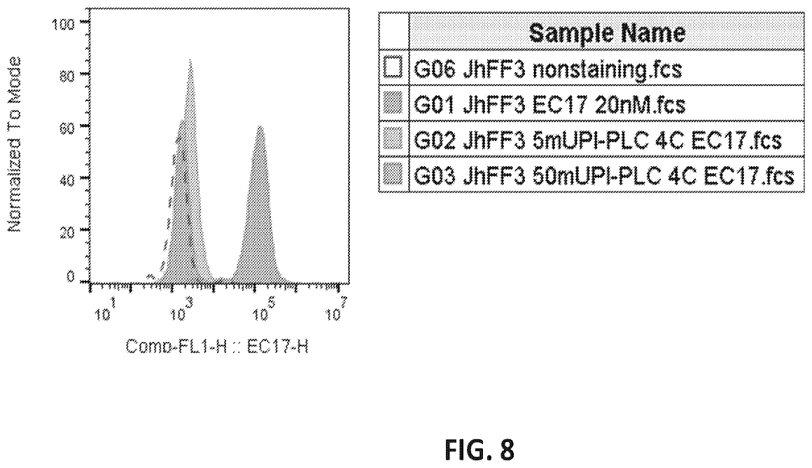

Using PI-PLC treat T cells having FF3 fusion protein resulted the release of GPI anchored receptor FF3. Jurkat T cell with FF3 fusion receptor shows saturated binding with 20 nM FA-FITC (EC17), while after 5 mU PI-PLC or 50 mU PI-PLC treatment, the FA-FITC loses binding to the cell, which indicates the release of the GPI anchored FF3 fusion receptor. See FIG. 8.

### 2.4 Fusion Protein FKBPFR3GS in Human T Cell Retains FR Binding Property

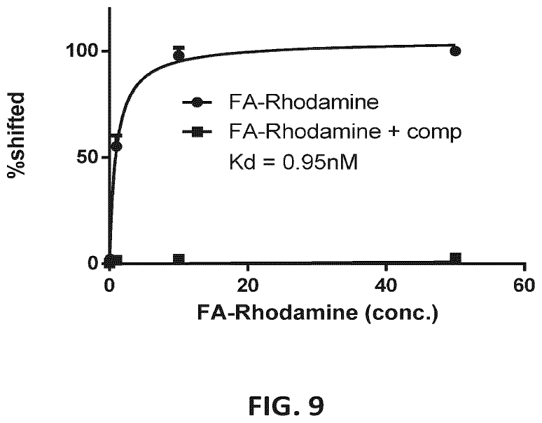

FA-Rhodamine binding curve is shown in FKBPFR3GS fusion receptor. FKBPFR3GS fusion receptor that stably expressed on human T cell can bind to folate acid derivative (FA-Rhodamine) with high affinity (Kd=0.95 nM), which is comparable to the affinity of FA-Rhodamine in FR+ KB cell (Kd around 1 nM). Therefore, the binding property of FR in the fusion receptor is preserved. See FIG. 9.

### 2.5 Fusion Protein FKBPFR3GS in Human T Cell Retains FKBP Binding Property

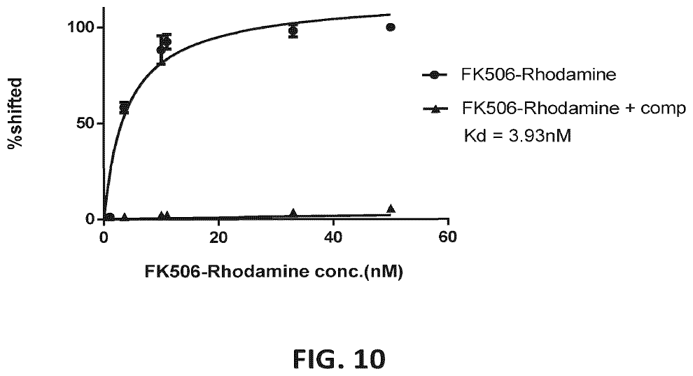

FK506-Rhodamine binding curve in FKBPFR3GS fusion receptor. FKBPFR3GS fusion receptor that stably expressed on human T cell is able to bind to FK506 derivative (FK506-Rhodamine) with high affinity (Kd=3.93 nM), which means the binding property of FKBP in the fusion receptor is preserved. See FIG. 10.

### 2.6 SLF-FITC Binds to FKBPFR3GS Fusion Receptor With Relative High Binding Affinity (Kd=62 nM)

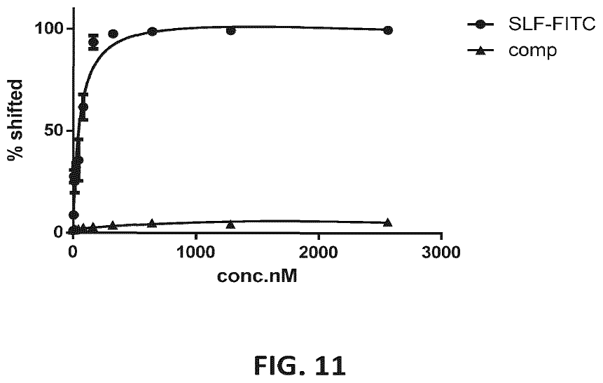

Competition of free SLF (100, preincubation) blocks SLF-FITC binding. SLF, a mimic of FK506, presents a 10 times lower binding affinity to FKBPFR fusion receptor, compared to the parent ligand FK506. See FIG. 11 and compare with FIG. 10.

### 2.7 Binding Curve of FA-Rhodamine in 4M5.3FR Fusion Receptor

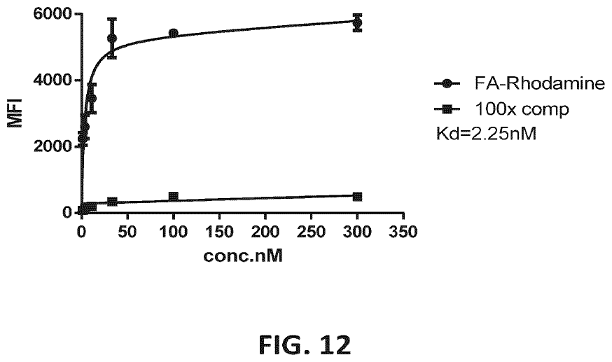

FA-Rhodamine can bind to 4M5.3FR fusion receptor that stably expressed on human T cell with high affinity (Kd=2.25 nM), which is comparable to the affinity of FA-Rhodamine in FR+ KB cell (Kd around 1 nM). Therefore, FR binding property is preserved in 4M5.3FR fusion receptor. See FIG. 12.

### 2.8 FITC-AF647 Binding Curve in 4M5.3FR Fusion Receptor

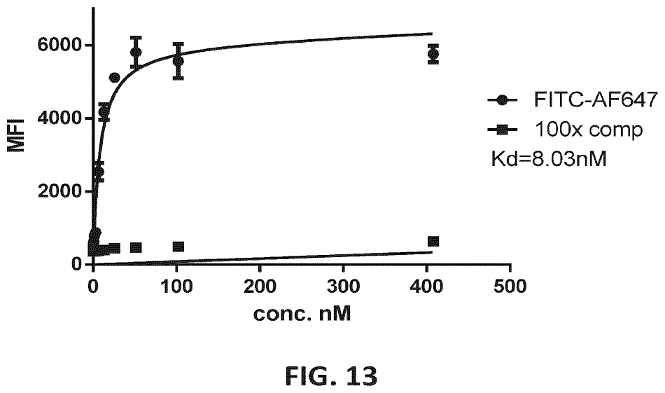

FITC-AF647 can bind to 4M5.3FR fusion receptor that stably expression on human T cell with high affinity (Kd=8.03 nM). 100× comp indicates free FITC sodium. The binding property of scFv 4M5.3 with FITC is preserved in 4M5.3FR fusion receptor. See FIG. 13.

### 3.1. FA-Tubulysin Killing Effect Against FF3+ Human T Cell

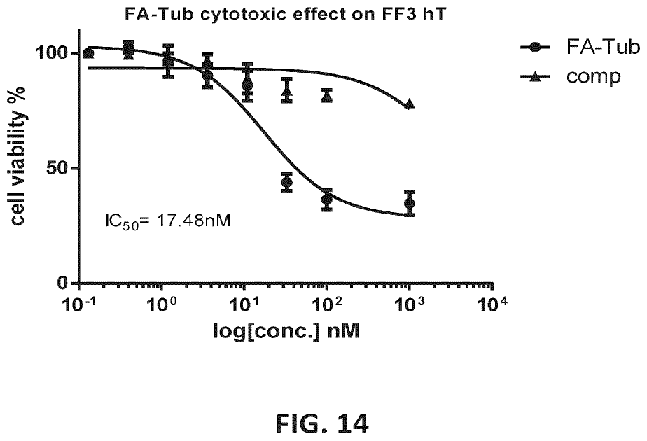

FA-Tubulysin is able to mediate a receptor specific killing effect against FF3+ human T cell. Compensation with FA (100× preincubation with FA) blocks the effect. This implies the successful internalization and release of the free drug Tubulysin through the FF3 fusion receptor system. See FIG. 14.

### 3.2 FA-Tubulysin Killing Effect Against hFF3 + Population in a Mixed Human T Cell Culture

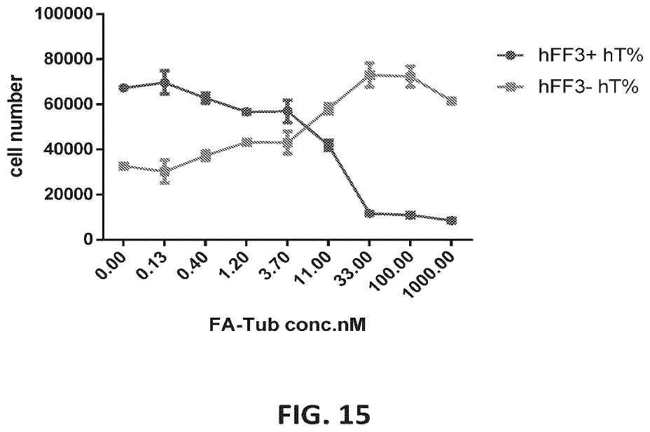

FA-Tubulysin specifically kills the hFF3+ population in a mixed human T cell culture. Percentage of hFF3+ cell decrease as the FA-Tub concentration increases. See FIG. 15.

### 3.3 SLF-Tub Specifically Kill the hFF3+ Jurkat Cells With a IC₅₀=138 nM

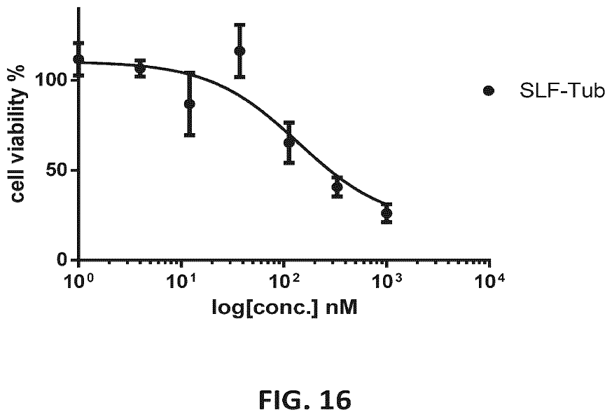

2 h incubation of SLF-Tub with hFF3 Jurkat cells is able to kill the receptor positive cells. This indicates the successful internalization of SLF-Tub by the FKBPFR3GS fusion receptor and the release of the Tubulysin inside the cell. See FIG. 16.

### 3.4. FITC-DM4 and FITC-Tub Killing Effects Against 4M5.3FR+ Human T Cells

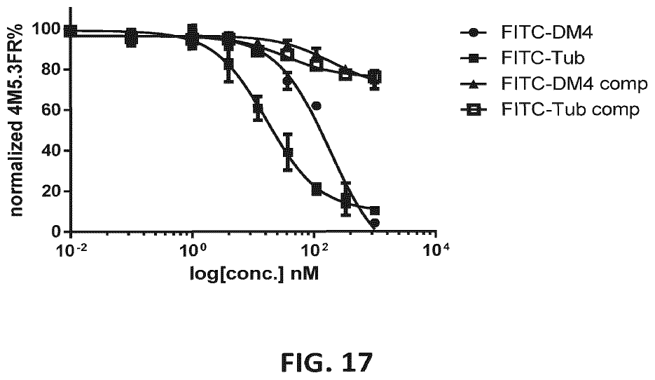

Both FITC-DM4 and FITC-Tub can specifically kill the 4M5.3FR+ human T cells, while FITC-Tub has a higher IC₅₀. Compensation of free FITC sodium (100× preincubation) blocks the receptor mediated killing effect. This implies the successful internalization and release of FITC-cytotoxic drug into T cell through 4M5.3FR fusion receptor. See FIG. 17.

### 3.5. FITC-Tubulysin Specifically Kill the 4M5.3FR+ Population in a Mixed Human T Cell Culture

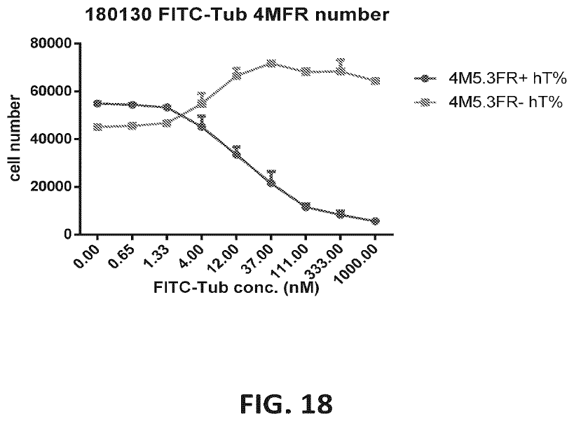

Absolute number of 4M5.3FR+ cell decrease as the FITC-Tub concentration increases, while 4M5.3FR− cells are killed also through released drugs and bystander effect at high concentration. See FIG. 18.

### 3.6. FITC-DM4 Specifically Kill the 4M5.3FR+ Population in a Mixed Human T Cell Culture

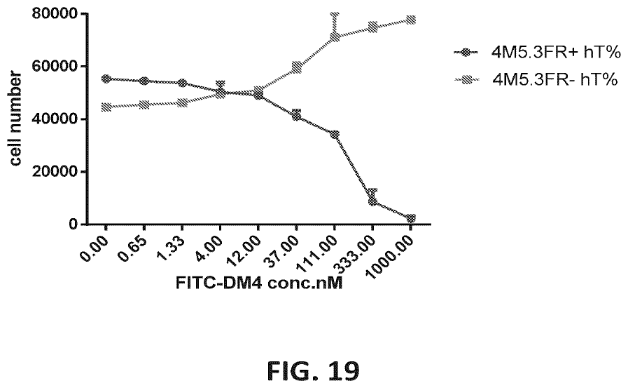

Absolute number of 4M5.3FR+ cell decrease as the FITC-DM4 concentration increases, while 4M5.3FR− cells are killed also through released drugs and bystander effect at high concentration. See FIG. 19.

### 3.7 Kinase Inhibitors Modulation Effect on Anti CD19 CAR T Cells Against CD19+ Raji

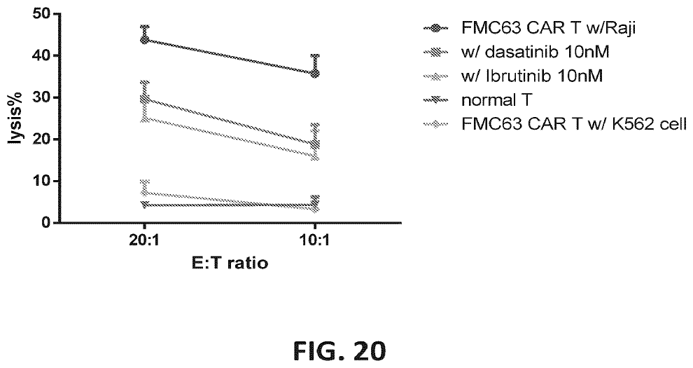

Dasatinib (Lck inhibitor) and Ibrutinib (ITK inhibitor) at 10 nM concentration decrease the lysis effect of antiCD19 CAR T cell (FMC63 CAR T, Effector) against CD 19+ Raji tumor cell (Target). Two Effector: Target ratio (E:T) have been tested. Normal T cell and antiCD19 CAR T with CD19− K562 cell were used as negative control. See FIG. 20.

### 3.8. FITC-Dasatinib Can Decrease the Lysis Effect of FMC63+4MFR+hT Cell Towards Raji Cell

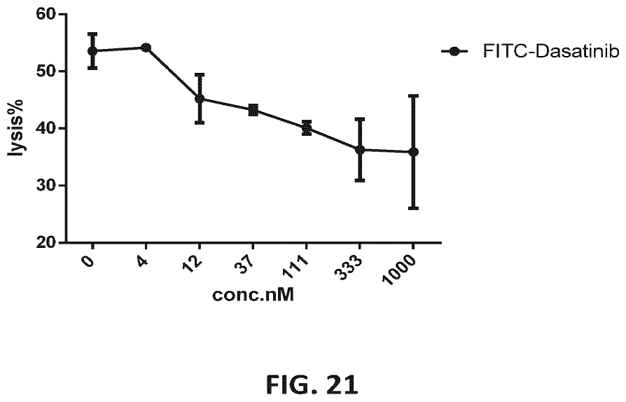

This implies the successful internalization and release of FITC-Dasatinib into T cell through 4M5.3FR fusion receptor and the release of Dasatinib into T cell. See FIG. 21.

### 3.9 TC-PTP Inhibitor at 100 nM Concentration Decrease the Co-Inhibitor Molecule Population in Exhausted antiCD19 CAR T Cell

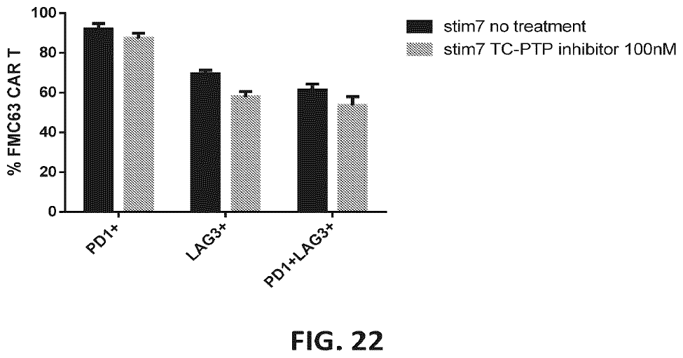

Exhausted antiCD19 CAR T cells are generated by 7 times of stimulation with CD19+Raji cells, see detailed procedure in material and methods section). PD-1 positive, LAG3 positive and double positive population decreases upon treatment. See FIG. 22.

### 4. Other FK506-Payload to Control the Activity of Cell Therapy

The technical advantageous feature of this drug payload delivery system is to have multi-functionality. The potential payloads and corresponding effects are listed below (Table 1). The small molecule payloads are selected based on the following parameters: 1. the functionality assay of the free drug, both in vitro and in vivo, has been confirmed by either published literature or work in our lab. 2. The chemical structure of the drug has relatively more accessible free amine for derivatization. 3. Any of the following will be preferable: FDA proved drug; commercially available for reasonable price. The FK506-payload will be tested first for in vitro experiments, T cell activation and stem cell cytokine release will be monitored by multiplex immunoassays. For in vivo disease models, we have well established CAR T therapy and bone fracture mouse models in our lab, and several potential collaborators for the neurodegenerative mouse models.

| Disease | | Cell | Cell | |
|---|---|---|---|---|
| Model | Subtype | Type | source | Payload type |
| stem cell | HSC transplant | BMSC | Murine BM | GSK3b inhibitor |
| therapy | | | | |
| Tumor | tumor | CAR T | hPBMC T | GSK3b inhibitor, |
| | microenvironment | | cell | HDAC inhibitor, |
| | | | | MAPK inhibitor |

1. A method to modulate cell therapy, comprising:
a. identifying a target cell for transplant, wherein the target cell has a cell therapy function;
b. providing an engineered protein on the surface of the target cell, wherein:
the engineered protein comprises a first component and a second component, the first component and the second component are connected by a peptide linker, the first component is a non-membrane protein, and the second component is a membrane anchored peptide or protein;
c. providing a conjugate comprising a payload drug to the target cell, wherein the payload drug is conjugated to a small molecule ligand through a linker, wherein the small molecule ligand binds to at least one component of the engineered protein with high affinity and is internalized by the target cell together with the payload drug; and
d. releasing the payload drug within the target cell to modulate the target cell therapy function.

2. The method according to claim 1, wherein the cell therapy function is to provide optically guided surgery to a subject.

3. The method according to claim 1, wherein the cell therapy function is to control the target cell proliferation.

4. The method according to claim 1, wherein the cell therapy function is to execute cytotoxicity to the target cell engaged cancer cell.

5. The method according to claim 1, wherein the target cell for transplant is an immune cell, a chimeric antigen receptor (CAR) T cell, a stem cell, a progenitor cell, or a transplanted cell designed to synthesize a biochemical that is deficient in a patient.

6. The method according to claim 1, wherein the payload drug is an imaging agent or a radioisotope imaging agent.

7. The method according to claim 6, wherein the imaging agent is selected from the group consisting of rhodamine, fluorescein, S0456, radioisotope chelating imaging moieties, EC 20 chelating head, NOTA, and DOTA.

8. The method according to claim 1, wherein the payload drug is a cytotoxic drug or a modulator of gene expression.

9. The method according to claim 8, wherein the payload drug is selected from an HDAC inhibitor, a kinase inhibitor, a metabolic inhibitor, tubulysin, DM1, DM4, an auristatin, a Dasatinib, a MEK1/2 inhibitor, a PI3 Kinase inhibitor, a GSK3 beta inhibitor, a MAO-B inhibitor, a Cdk5 inhibitor, an RORγt agonist, and an siRNA.

10. The method according to claim 1, wherein the target cell for transplant comprises a fusion protein comprising the sequence of amino acids set forth in any one of SEQ ID NOS: 1, 2, or 12-15.

11. The method according to claim 1, wherein the first component and/or the second component are independently selected from the group consisting of FRa, FRb, uPAR, FKBP, DHFR, scFv against FITC, and scFv against DNP.

12. The method according to claim 1, wherein the small molecule ligand is selected from the group consisting of FK506, FK506 derivatives, synthetic ligand of FKBP (SLF), SLF derivatives, folic acid (FA), and FA derivatives.

13. The drug delivery platform according to claim 12, wherein the small molecule ligand is FK506 or its derivative.

14. The method according to claim 1, wherein the linker to connect the small molecule ligand and the payload drug is selected from the group consisting of:

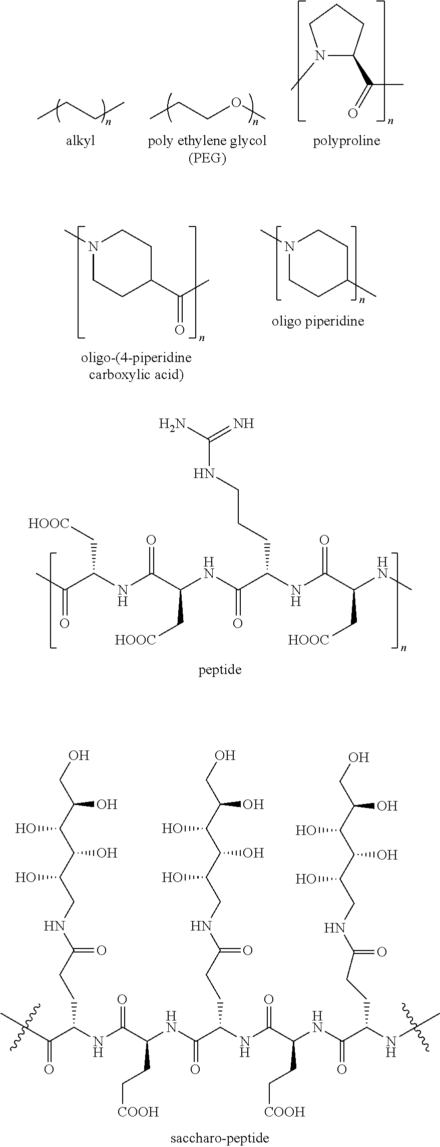

15. The method according to claim 1, wherein the target cell for transplant is a CAR T cell.

16. The method according to claim 1, wherein the conjugate comprises a FK506-releasable linker of formula I:

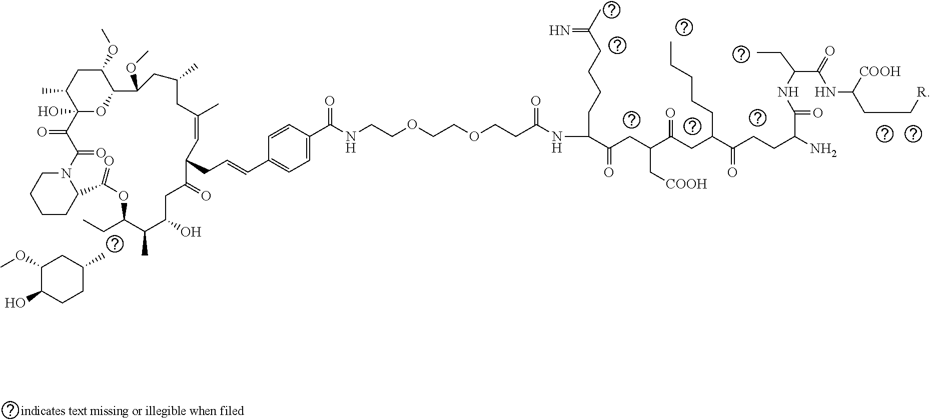

17. The method according to claim 1, wherein the target cell for transplant is a CAR T cell and the conjugate is designed to control a cytokine storm induced by the CAR T cell, or contains a modulator designed to control unwanted T cell proliferation.

18. The method according to claim 1, wherein the target cell for transplant is a NK cell and the conjugate is a RORγt agonist to control Th17 cell mediated immune responses.

19. The method according to claim 1, wherein the payload drug is a phosphatase inhibitor.

20. The method according to claim 19, wherein the phosphatase inhibitor is an inhibitor against SHP1/2 or TC-PTP.


SEQUENCE LISTING (22 sequences)

SEQ ID NO: 1 - protein, 367 aa, synthetic construct
  REGION 1..367: note = A synthetic amino acid sequence for mouse FKBP-FRa with linker peptide
MAHLMTVQLLLLVMWMAECAQSRAGVQVETISPGDGRTFPKRGQTCVVHYTGMLEDGKKFDSSRDRNKPFKFTLGKQEVIRGWEEGVAQMSVGQRAKLIISSDYAYGATGHPGIIPPHATLVFDVELLKLESGGGSTRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 2 - protein, 369 aa, synthetic construct
  REGION 1..369: note = A synthetic amino acid sequence for human FKBP-FRa with linker peptide
MAQRMTTQLLLLLVWVAVVGEAQTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 3 - protein, 485 aa, synthetic construct
  REGION 1..485: note = A synthetic amino acid sequence for mouse antiCD19 CAR T construct
MGVPTQLLGLLLLWITDAICEQKLISEEDLDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 4 - protein, 496 aa, synthetic construct
  REGION 1..496: note = A synthetic amino acid sequence for human antiCD19 CAR T construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - DNA, 12182 bp, synthetic construct
  misc_feature 1..12182: note = A synthetic vector sequence for pWPI for human T cell transduction
  source 1..12182: mol_type = other DNA
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttatggctcacctgatgactgtgcagttgttgctcctggtgatgtggatggccgaatgtgctcagtccagagctggagtgcaggtggagaccatctctcctggagacgggcgcaccttcccaaagcgcggccagacctgcgtggtgcactacacggggatgcttgaagatggaaagaaatttgattcctctcgggacagaaacaagccttttaagtttacactaggcaagcaggaggtgatccgaggctgggaggaaggggtagcccagatgagtgtgggtcagagagccaaactgataatctcctcagactatgcctatggagccaccgggcacccaggcatcatcccaccacatgccactcttgtttttgatgtggagcttctaaaactggaaagcggcggcggcagcactcgggccaggactgaacttctcaatgtctgcatggatgccaaacaccacaaagaaaaaccgggccctgaggacaatttacacgaccagtgcagcccctggaagacgaattcctgctgttccacgaacacaagccaggaagcacataaggacatttcctacctgtaccggttcaactggaaccactgcggaactatgacatcggaatgcaaacggcactttatccaagacacctgcctctatgagtgttccccgaacttgggaccctggatccagcaggtggaccagagctggcgcaaagagcggatccttgatgttcccctgtgcaaagaggactgtcagcagtggtgggaggactgccagagctcttttacctgcaagagcaattggcacaagggatggaactggtcctcggggcataacgagtgtcctgtgggagcctcctgccatcccttcaccttctacttccccacatctgctgctctgtgtgaggaaatctggagtcactcctacaagctcagcaactacagtcgagggagcggccgctgcattcagatgtggttcgacccagcccagggcaaccccaacgaggaagtggcgaggttctatgccgaggccatgagtggagctgggtttcatgggacctggccactcttgtgcagcctgtccttagtgctgctctgggtgatcagctgaaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 6 - DNA, 6939 bp, synthetic construct
  misc_feature 1..6939: note = A synthetic pMP71 gb NotIEcoRI mouse antiCD19 for mouse T cell transduction
  source 1..6939: mol_type = other DNA
tcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgggcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcatgggggttcccacccagctgttggggttgctgctcctctggatcactgatgccatctgcgaacagaagctcatttctgaagaagatctggacatacagatgacccagagtcctgccagcttgtccacatccctcggagagaccgtgacaattcaatgccaggcctctgaggacatatattctggattggcttggtaccagcaaaagccaggtaaaagtcctcagttgttgatatacggcgcttctgatttgcaagacggggtgccctcacgatttagcgggtctggaagtggcactcagtacagtctgaagattacttcaatgcaaacagaagacgagggtgtgtatttttgccaacaggggctgacctacccaaggacattcgggggcggtacaaagcttgaacttaagggcggcggtgggtctggaggtggtggatctggcggagggggaagtgaggtacagctgcaacagtccggcgccgaactcgttcgccctggaacctcagtcaaattgtcatgcaaggtgagtggcgacacaataaccttttactatatgcacttcgttaaacagaggccaggtcaaggtctggaatggataggcagaattgatccagaagatgagtccaccaaatactcagaaaagttcaaaaacaaagccactcttactgccgacacctcaagcaacacagcatatcttaagctcagttcacttaccagcgaagacaccgccacctatttttgtatttatggtggctattactttgactactgggggcaaggtgttatggtaacagtttcttccattgaattcatgtatccacccccctatttggataatgaacgatctaatgggactataatacatatcaaggaaaagcatctgtgtcatacccaaagttcccctaagcttttctgggccctcgtcgttgtggcaggagtgcttttttgctatggattgttggttactgtggctctctgcgtcatttggacaaatagtaggaggaatcgggggggacaatctgattacatgaacatgacaccacggaggccaggccttaccagaaagccctaccaaccttatgcaccagcacgagacttcgccgcatacaggccaagggctaagttttcccgcagcgccgaaaccgcagccaacctccaagatcctaatcagctctataacgaattgaatcttggccgcagagaggagtacgacgtacttgagaaaaagagagctagggaccctgaaatgggtgggaagcaacagcgaagaaggaacccacaggaaggggtgtataatgcccttcaaaaggataaaatggcagaggcatacagtgaaatcggaaccaagggggagagacgcagagggaaaggccatgacggcctttatcagggtttgtcaactgctactaaagacacttatgatgccttgcatatgcaaactctcgcacccagatgacgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttcctaacacgagccatagatagaataaaagattttatttagtctccagaaaaaggggggaatgaaagaccccacctgtaggtttggcaagctagcttaagtaagccattttgcaaggcatggaaaaatacataactgagaatagagaagttcagatcaaggttaggaacagagagacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccggggtacccgtattcccaataaagcctcttgctgtttgcatccgaatcgtggactcgctgatccttgggagggtctcctcagattgattgactgcccacctcgggggtctttcattctcgagagctttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgctggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagttactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaagccattttgcaaggcatggaaaaatacataactgagaatagagaagttcaga

SEQ ID NO: 7 - DNA, 12188 bp, synthetic construct
  misc_feature 1..12188: note = A synthetic pWPI-FRa 1-24 FKBP FRa
  source 1..12188: mol_type = other DNA
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttatggctcagcggatgacaacacagctgctgctccttctagtgtgggtggctgtagtaggggaggctcagacaggagtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctgaaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 8 - DNA, 12182 bp, synthetic construct; identical to SEQ ID NO: 5
  misc_feature 1..12182: note = A synthetic pWPI mFKBP-mFRa SGGGS
  source 1..12182: mol_type = other DNA

SEQ ID NO: 9 - DNA, 10447 bp, synthetic construct
  misc_feature 1..10447: note = A synthetic pHR EcorI hAnti cd19 1D3 myc hinge cd28 cd3zeta
  source 1..10447: mol_type = other DNA
cgataccgtcgaccaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgatggctcttccagtgactgctcttctcctcccgcttgcgctgctgttgcatgcggctcggccggagcaaaagctgatttcagaagaggacttggatatccagatgacacagaccacttcatctctttctgctagcctgggggatcgggtcacaataagctgtcgcgcatcccaagacataagcaaatatctgaattggtatcaacagaaacccgatggaactgtgaaacttctcatctaccatacgagcagactgcattctggggttcctagccgcttttcagggtctggatctggaacggactattcacttaccatatctaatttggaacaagaagacatcgcgacctatttctgtcagcaaggcaatacgctcccttatacttttggagggggaacgaagttggagatcacaggtggaggcggcagtggcgggggaggatctggtggaggtggttctgaggtcaagctgcaggagagtggtcccgggctggtagccccgagccagagtctgtctgttacttgcactgtgtcaggcgtgagtctcccagactatggtgtatcatggattcgacagccgccccggaaaggacttgagtggctcggagtgatctggggatccgaaacgacgtactacaatagcgcgctcaaaagccggctgaccatcattaaggataactctaaaagccaggtgttcttgaaaatgaattccttgcagacagatgatacggcgatctattactgtgccaagcactactactatggaggcagctatgccatggattattggggtcaaggcacttctgtgacagtgagcagtgccgcagctattgaagtgatgtacccgcctccgtatcttgataatgagaaatctaacggaaccataatacacgtgaaaggcaaacatttgtgtccgtctcctctgttccccgggcctagtaaaccgttttgggtactggtggtggtaggcggagtacttgcatgttactcactcctcgttaccgtcgcattcattatcttctgggtacggagcaagagatctcggctgcttcatagcgattatatgaatatgacacccagacgcccaggtcctactaggaaacattaccagccgtatgcgccgccgagggacttcgccgcatacagatctcgcgtgaagttctcaagatctgccgatgcaccggcttatcagcagggacaaaaccaactgtataatgagctgaacctggggcggagggaggagtatgatgtcctggacaagcgaagagggcgagaccccgaaatgggaggcaagcctcaacggcggaagaacccacaagagggcctgtataacgagcttcaaaaggacaaaatggcggaagcgtatagcgagatcggaatgaagggcgaacgaaggagagggaaaggtcacgatgggctctaccaggggctcagcacggcaacaaaagacacatatgacgcattgcatatgcaggcgctgccaccgagatgactcacgcgtcaagtggagcaaggcaggtggacagtggatccttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 10 - DNA, 12536 bp, synthetic construct
  misc_feature 1..12536: note = A synthetic pWPI pmei mAnti cd19 1D3 myc hinge cd28 cd3zeta
  source 1..12536: mol_type = other DNA
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttatgggggttcccacccagctgttggggttgctgctcctctggatcactgatgccatctgcgaacagaagctcatttctgaagaagatctggacatacagatgacccagagtcctgccagcttgtccacatccctcggagagaccgtgacaattcaatgccaggcctctgaggacatatattctggattggcttggtaccagcaaaagccaggtaaaagtcctcagttgttgatatacggcgcttctgatttgcaagacggggtgccctcacgatttagcgggtctggaagtggcactcagtacagtctgaagattacttcaatgcaaacagaagacgagggtgtgtatttttgccaacaggggctgacctacccaaggacattcgggggcggtacaaagcttgaacttaagggcggcggtgggtctggaggtggtggatctggcggagggggaagtgaggtacagctgcaacagtccggcgccgaactcgttcgccctggaacctcagtcaaattgtcatgcaaggtgagtggcgacacaataaccttttactatatgcacttcgttaaacagaggccaggtcaaggtctggaatggataggcagaattgatccagaagatgagtccaccaaatactcagaaaagttcaaaaacaaagccactcttactgccgacacctcaagcaacacagcatatcttaagctcagttcacttaccagcgaagacaccgccacctatttttgtatttatggtggctattactttgactactgggggcaaggtgttatggtaacagtttcttccattgaattcatgtatccacccccctatttggataatgaacgatctaatgggactataatacatatcaaggaaaagcatctgtgtcatacccaaagttcccctaagcttttctgggccctcgtcgttgtggcaggagtgcttttttgctatggattgttggttactgtggctctctgcgtcatttggacaaatagtaggaggaatcgggggggacaatctgattacatgaacatgacaccacggaggccaggccttaccagaaagccctaccaaccttatgcaccagcacgagacttcgccgcatacaggccaagggctaagttttcccgcagcgccgaaaccgcagccaacctccaagatcctaatcagctctataacgaattgaatcttggccgcagagaggagtacgacgtacttgagaaaaagagagctagggaccctgaaatgggtgggaagcaacagcgaagaaggaacccacaggaaggggtgtataatgcccttcaaaaggataaaatggcagaggcatacagtgaaatcggaaccaagggggagagacgcagagggaaaggccatgacggcctttatcagggtttgtcaactgctactaaagacacttatgatgccttgcatatgcaaactctcgcacccagatgaaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 11 - protein, 365 aa, synthetic construct
  REGION 1..365: note = A synthetic FKBP-1SG-FR with GPI anchor amino acid sequence
MTTQLLLLLVWVAVVGEAQTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 12 - protein, 375 aa, synthetic construct
  REGION 1..375: note = A synthetic FKBP-3SG-FR with GPI anchor amino acid sequence
MTTQLLLLLVWVAVVGEAQTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSGGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 13 - protein, 513 aa, synthetic construct
  REGION 1..513: note = A synthetic 4M5.3-FR with GPI anchor amino acid sequence
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSSGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 14 - protein, 895 aa, synthetic construct
  REGION 1..895: note = A synthetic FMC63-T2A-FKBP3SGFR
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAQRMTTQLLLLLVWVAVVGEAQTGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSGGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 15 - protein, 1029 aa, synthetic construct
  REGION 1..1029: note = A synthetic FMC63-T2A-4M5.3SGFR
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSSGGGSRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 16 - protein, 255 aa, synthetic construct
  REGION 1..255: note = A synthetic FRb with signal peptide
MVWKWMPLLLLLVCVATMCSAQDRTDLLNVCMDAKHHKTKPGPEDKLHDQCSPWKKNACCTASTSQELHKDTSRLYNFNWDHCGKMEPACKRHFIQDTCLYECSPNLGPWIQQVNQSWRKERFLDVPLCKEDCQRWWEDCHTSHTCKSNWHRGWDWTSGVNKCPAGALCRTFESYFPTPAALCEGLWSHSYKVSNYSRGSGRCIQMWFDSAQGNPNEEVARFYAAAMHVNAGEMLHGTGGLLLSLALMLQLWLLG

SEQ ID NO: 17 - protein, 335 aa, synthetic construct
  REGION 1..335: note = A synthetic uPAR with signal peptide
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 18 - protein, 187 aa, synthetic construct
  REGION 1..187: note = A synthetic DHFR
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 19 - protein, 255 aa, synthetic construct
  REGION 1..255: note = A synthetic scFv against FITC: 4M5.3(Kd = 200pM)
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSS

SEQ ID NO: 20 - protein, 244 aa, synthetic construct
  REGION 1..244: note = A synthetic scFv against FITC 4D5Flu (Kd=10nM)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 21 - protein, 244 aa, synthetic construct
  REGION 1..244: note = A synthetic scFv against DNP SPE7
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVLGGGGSGGGGSGGGGSEVQLQQPGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSS

SEQ ID NO: 22 - protein, 4 aa, synthetic construct
  REGION 1..4: note = A synthetic sequence
DDRD